(19) United States
(12) Patent Application Publication
(10) Pub. No.: US 2021/0069214 A1
(43) Pub. Date: Mar. 11, 2021

(54) TOPICAL COMPOSITIONS COMPRISING A CORTICOSTEROID FOR THE TREATMENT OF PSORIASIS IN PEDIATRIC PATIENTS

(71) Applicant: ENCORE DERMATOLOGY, INC., Malvern, PA (US)

(72) Inventor: Srinivas SIDGIDDI, Princeton, NJ (US)

(73) Assignee: ENCORE DERMATOLOGY, INC., Malvern, PA (US)

(21) Appl. No.: 17/013,293

(22) Filed: Sep. 4, 2020

Related U.S. Application Data
(60) Provisional application No. 62/896,723, filed on Sep. 6, 2019.

(51) Int. Cl. A61K 31/575; A61K 9/00; A61K 47/24; A61K 47/10; A61K 47/14; A61K 47/12; A61K 47/08; A61K 47/44; A61P 17/06

(52) U.S. Cl. CPC A61K 31/575; A61K 9/0014; A61K 47/24; A61K 47/10; A61P 17/06; A61K 47/12; A61K 47/08; A61K 47/44; A61K 47/14

(57) ABSTRACT

The present invention relates to topical compositions comprising a corticosteroid and at least one penetration enhancing agent, wherein the composition is substantially free of propylene glycol for use in the treatment of psoriasis in pediatric patients.

### CROSS-REFERENCE TO RELATED APPLICATIONS

This application claims the benefit of priority to U.S. Provisional Application 62/896,723 entitled “Topical Compositions Comprising a Corticosteroid for the Treatment of Psoriasis in Pediatric Patients,” filed Sep. 6, 2019, which is incorporated herein by reference in its entirety.

### TECHNICAL FIELD

The present invention relates to a method comprising topically administering to pediatric subjection a composition comprising clobetasol and at least one penetration enhancing agent. The composition of the present invention is results in a decreased risk of hypothalamic-pituitary-adrenal (HPA) axis suppression, a decreased risk of side effects, or a combination thereof.

### BACKGROUND

Topical corticosteroids are the most frequently prescribed drugs by dermatologists for treating psoriasis, relief of the inflammatory and pruritic manifestations of steroid responsive dermatoses, and associated diseases or disorders. The corticosteroids are a class of compounds comprising steroids (lipids that contain a hydrogenated cyclopentoperhydrophenanthrene ring system) elaborated by the adrenal cortex (except sex hormones of adrenal origin) in response to the release of adrenocorticotrophin or adrenocorticotropic hormone by the pituitary gland, or to any synthetic equivalent, or to angiotensin II. In pharmacologic doses, corticosteroids are used primarily for their anti-inflammatory and/or immunosuppressive effects.

Topical corticosteroids, such as clobetasol propionate, are effective in treatment of corticosteroid-responsive dermatoses primarily because of their anti-inflammatory, antipruritic and vasoconstrictive actions. Clobetasol propionate is used to treat various other skin disorders including eczema and psoriasis. It is also highly effective for contact dermatitis caused by exposure to poison ivy/oak.

Clobetasol propionate is chemically known as [17-(2′-chloroacetyl)-9-fluoro-11-hydroxy-10,13,16-trimethyl-3-oxo-6,7,8,11,12,14,15,16-octahydrocyclopenta[a]phenanthren-17-yl]propanoate and is represented by structural Formula I:

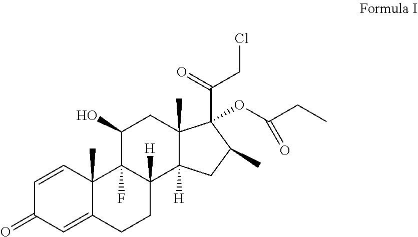

Clobetasol propionate is commercially available in compositions for topical application in the form of aerosol foam, cream, ointment, gel, solution, lotion, spray or shampoo, in a weight concentration of 0.05%. TEMOVATE® cream is a commercially available product of clobetasol approved by U.S. Food and Drug Administration (FDA) on Dec. 27, 1985 and is currently being marketed by Fougera Pharms. TEMOVATE® cream contains Clobetasol propionate 0.5 mg/g in a cream base of propylene glycol, glyceryl monostearate, cetostearyl alcohol, glyceryl stearate, PEG 100 stearate, white wax, chlorocresol, sodium citrate, citric acid monohydrate and purified water. TEMOVATE® E is another approved product by U.S. Food and Drug Administration (FDA) containing Clobetasol propionate (0.05% (w/w)) in a cream base of cetostearyl alcohol, isopropyl myristate, propylene glycol, ceteth-20, dimethicone 350, citric acid monohydrate, sodium citrate, imidurea, and purified water.

U.S. Pat. No. 5,972,920 is related to a formulation characterized by a carrier compound formed of a combination of two components in a volume ratio of about 50/50, wherein a first carrier component is selected from the group consisting essentially of ethyl alcohol and isopropyl alcohol and a second carrier component is selected from the group consisting essentially of isopropyl myristate, isopropyl palmitate, octyl palmitate, octyl isononanoate, and isocetyl stearate. The formulation also comprises an anionic surfactant.

PCT Application WO 2006/115987 is related to a method for treating psoriasis by spraying a pharmaceutical composition containing an effective amount of clobetasol propionate onto the skin with psoriasis, using a daily treatment for at least 4 weeks. The preferred composition is a spray formulation of clobetasol propionate 0.05%, containing alcohol, isopropyl myristate, an anionic surfactant such as sodium lauryl sulfate, and optionally, an antimicrobial compound such as an antifungal compound, e.g., undecylenic acid.

U.S. Pat. Nos. 6,419,913 and 6,284,234 are related to topical delivery systems for active agents comprising micellar compositions.

U.S. Publication No. 2006/0099173 is related to a process of making a pharmaceutical composition for topical application, the composition being an emulsion comprising water and at least one active ingredient.

U.S. Publication No. 2007/0142343 is related to a composition comprising corticosteroids, penetration enhancers, solvents and emulsifiers. The vehicle of this composition utilizes at least two penetration enhancers, including diisopropyl adipate, dimethyl isosorbide, propylene glycol, 1,2,6-hexanetriol, and benzyl alcohol.

US publication No. 2009/0104131 is related to a topically applicable compositions in the form of oil-in-water (O/W) emulsions contain a pro-penetrating system including at least one glycol and at least one additional pro-penetrating agent, a suitable emulsifying system and at least one active agent of the family of steroidal anti-inflammatory agents. Propylene glycol is disclosed as pro-penetrating agent.

U.S. Pat. No. 6,579,512 is related to topical spray composition comprising clobetasol propionate, ethanol, propellant and isopropyl myristate.

U.S. Pat. Nos. 7,700,081 and 7,316,810 are related to clobetasol propionate (0.05 wt %) shampoo compositions used for washing and treating the ailments of scalp.

Dermatological corticosteroids, in particular clobetasol propionate topical preparations face multiple problems, such as delivery efficiency, stability, and tolerability, in particular with respect to excipients that would not cause irritation. In addition, corticosteroids can be absorbed through the skin and can cause systemic side effects, for example hypothalamic pituitary adrenal (HPA) axis suppression. Therefore, to avoid unwanted side effects, the corticosteroid is used at a concentration as low as possible. However, topical preparations containing low concentrations corticosteroids cannot ensure a sufficient therapeutic effect.

U.S. Publication No. 2010/0249060 is related to a low dose clobetasol propionate composition in aqueous vehicle based on propylene glycol and macrogol-glycerol hydroxysterate.

Although several of the above noted references disclose clobetasol propionate containing compositions, most of them are greasy, and hence are unpleasant to apply on large areas of the skin. In addition, some conventional cream and ointment bases containing propylene glycol are irritating to the skin, particularly over the long exposure that is frequently required for efficacy. The fluidity of lotions often makes their physical application difficult to control over a desired area. Further, formulations containing ethanol or propylene glycol may be associated with an elevated risk of sensitization and have a tendency to induce irritation, and thus, such formulations do not promote patient compliance. The currently available topical compositions comprising clobetasol appears to show adverse effect on endocrine system as described in TEMOVATE® cream and TEMOVATE® E cream labels (Hypothalamic-pituitary-adrenal axis suppression).

While topical corticosteroids, such as clobetasol propionate, are effective in treatment of corticosteroid-responsive dermatoses such as psoriasis in adults, clobetasol use in pediatric patients is not recommended and can lead to a higher incidence of adverse effects than in adult patients. IMPOYZ™ is not currently approved for use in patients under the age of 18 as children may be more susceptible to systemic toxicity from use of topical corticosteroids. Until now, the safety and effectiveness of IMPOYZ™ Cream in patients younger than 18 years of age had not been established; therefore, use in children younger than 18 years could not be recommended. Because of a higher ratio of skin surface area to body mass, pediatric patients are at a greater risk than adults of systemic toxicity, including HPA axis suppression.

Accordingly, there is a long felt need to develop effective topical clobetasol composition for pediatric subjects with reduced concentration of active, but having an effect comparable to that obtainable with conventional topical clobetasol propionate compositions. Further it is desirable to have a clobetasol propionate composition with improved absorption without causing any skin irritation.

### SUMMARY OF THE INVENTION

Described herein are methods comprising topically administering twice daily for up to about two weeks to a pediatric subject a composition comprising about 0.05% to about 0.045% (w/w) of clobetasol, an aqueous phase, an oil phase; about 5% of an emollient, about 0.05% of an antioxidant, about 10% of a solvent, about 6% of an emulsifier, and a preservative; wherein the aqueous phase is water and the water is at least 60% the total weight of the composition, wherein the oil phase comprises at least one penetration enhancing agent in an amount from about 0.01% to about 15.0% of the total weight of the composition and a non-polymeric thickening agent, wherein the pediatric subject has a risk of HPA axis suppression that is similar to the risk of HPA axis suppression in an adult subject.

In some embodiments, the composition is topically administered to a pediatric subject with moderate to severe plaque psoriasis. In some embodiments, the pediatric subject has moderate to severe psoriatic lesions involving at least about 10% body surface area. In some embodiments, the pediatric subject has an investigator global assessment score of greater than or equal to 3. In some embodiments, treating moderate to severe plaque psoriasis in the subject results in an investigator global assessment score of 0 to 1. In some embodiments, the investigator global assessment score is measured at day 15.

In some embodiments, the pediatric subject is a male or female aged about 6 to about 16 years and 11 months of age. In some embodiments, the pediatric subject is a male or female aged about 12 to about 16 years and 11 months of age. In some embodiments, the pediatric subject is a male or female under the age of 18 years. In some embodiments, the pediatric subject is a male or female under the age of 12 years.

In some embodiments, topical administration of the composition to a pediatric subject results in an at least 80% chance that the subject will not develop hypothalamic-pituitary-adrenal (HPA) axis suppression. In some embodiments, topical administration of the composition to a pediatric subject results in plasma concentrations of clobetasol that are insufficient to reduce serum cortisol levels less than or equal to 18 μg/dL. In some embodiments, topical administration of the composition to a pediatric subject results in the subject being substantially free of adverse effects. In further embodiments the adverse effect is selected from skin irritation, vein collapse, itching, burning, stinging and a combination thereof. In further embodiments the adverse effect is selected from Cushing's syndrome, hyperglycemia and glycosuria, linear growth retardation, delayed weight gain, and intracranial hypertension and a combination thereof. In some embodiments, topical administration to a pediatric subject results in no HPA axis suppression. In some embodiments, topical administration in a pediatric subject results in substantially no HPA axis suppression.

In some embodiments, the clobetasol is clobetasol proprionate. In some embodiments, the topical administration of the composition provides a mean clobetasol plasma level less than about 10 pg/mL.

In some embodiments, the emollient comprises cyclomethicone. In some embodiments, the antioxidant comprises butylated hydroxytoluene. In some embodiments, the solvent comprises isopropyl myristate. In some embodiments, the emulsifier comprises glyceryl stearate and PEG 100 stearate. In some embodiments, the preservative comprises one or both of methylparaben and propylparaben. In further embodiments, the preservative is methylparaben and the methylparaben is about 0.2% of the total weight of the composition. In further embodiments, the preservative is propylparaben and propylparaben is about 0.4% of the total weight of the composition. In some embodiments, the penetration enhancing agent is diethylene glycol monoethyl ether. In some embodiments, the non-polymeric thickening agent comprises one or both of cetosteryl alcohol and white wax. In some embodiments, the oil phase comprises at least one penetration enhancing agent in an amount up to about 5.0% of the total weight of the composition, wherein the at least one penetration enhancing agent is diethylene glycol monoethyl ether. In some embodiments, the oil phase comprises at least one penetration enhancing agent in an amount of about 3.0% of the total weight of the composition, wherein the at least one penetration enhancing agent is diethylene glycol monoethyl ether.

In some embodiments, the topically administered composition is substantially free of propylene glycol.

In some embodiments, the topically administered composition is substantially free of polymers.

### DETAILED DESCRIPTION OF THE INVENTION

The details of one or more embodiments of the present invention are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Topical corticosteroids are the most frequently prescribed drugs by dermatologists for treating psoriasis, relief of the inflammatory and pruritic manifestations of steroid responsive dermatoses, and associated diseases or disorders. The corticosteroids are a class of compounds comprising steroids (lipids that contain a hydrogenated cyclopentoperhydrophenanthrene ring system) secreted by the adrenal cortex (except sex hormones of adrenal origin) in response to the release of adrenocorticotrophin or adrenocorticotropic hormone by the pituitary gland, or to any synthetic equivalent, or to angiotensin II. In pharmacologic doses, corticosteroids are used primarily for their anti-inflammatory and/or immunosuppressive effects.

Topical corticosteroids, such as clobetasol propionate, are effective in treatment of corticosteroid-responsive dermatoses primarily because of their anti-inflammatory, antipruritic and vasoconstrictive actions. Clobetasol propionate is used to treat various other skin disorders including eczema and psoriasis. It is also highly effective for contact dermatitis caused by exposure to poison ivy/oak.

Clobetasol propionate is chemically known as [17-(2′-chloroacetyl)-9-fluoro-11-hydroxy-10, 13,16-trimethyl-3-oxo-6,7,8, 11,12,14,15,16-octahydrocyclopenta[a]phenanthren-17-yl]propanoate and is represented by structural Formula I:

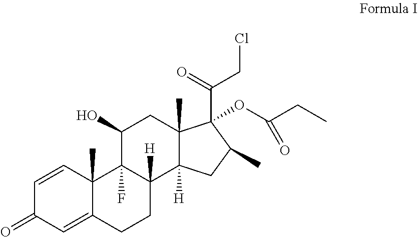

Dermatological corticosteroids, in particular clobetasol propionate topical preparations face multiple problems, such as delivery efficiency, stability, and tolerability, in particular with respect to excipients that would not cause irritation. In addition, corticosteroids can be absorbed through the skin and can cause systemic side effects, for example hypothalamic-pituitary-adrenal (HPA) axis suppression. Therefore, to avoid unwanted side effects, the corticosteroid is used at a concentration as low as possible. However, topical preparations containing low concentrations corticosteroids cannot ensure a sufficient therapeutic effect. Due to the risk of adverse effects and HPA axis suppression, corticosteroids are rarely used in pediatric patient populations. It has now been shown that topical compositions of clobetasol such as those described herein can be used safely in pediatric subjects.

IMPOYZ™ is a cream composition comprising 0.025% clobetasol propionate for the treatment of moderate to severe plaque psoriasis in patients 18 years of age or older. Rare systemic toxicities such as Cushing's syndrome, hyperglycemia, and glycosuria, linear growth retardation, delayed weight gain, and intracranial hypertension have been reported in pediatric patients, especially those with prolonged exposure to large doses of high potency topical corticosteroids. Local adverse reactions including striae and skin atrophy have also been reported with use of topical corticosteroids in pediatric patients. Systemic absorption of clobetasol propionate has been shown to suppress the HPA axis. Manifestations of adrenal suppression in children include low plasma cortisol levels and absence of response to ACTH stimulation. Manifestations of intracranial hypertension include bulging fontanelles, headaches, and bilateral papilledema. IMPOYZ™ is not currently approved for use in patients under the age of 18 as children may be more susceptible to systemic toxicity from use of topical corticosteroids. Until now, the safety and effectiveness of IMPOYZ™ Cream in patients younger than 18 years of age had not been established; therefore, use in children younger than 18 years could not be recommended. Because of a higher ratio of skin surface area to body mass, pediatric patients are at a greater risk than adults of systemic toxicity, including HPA axis suppression, when treated with topical drugs.

The highest approved topical dose of clobetasol propionate by U.S. Food and drug administration (U.S. FDA) for the treatment of one or more of skin disorders and the highest approved topical dose of clobetasol propionate by US FDA is 0.05% (w/w), i.e. TEMOVATE® or TEMOVATE® E. The term “TEMOVATE®” or TEMOVATE® E is used interchangeably for indicating “highest approved topical dose of clobetasol propionate’ i.e. 0.05% (w/w) in cream or gel or ointment or solution form; or its pharmaceutical equivalents or its therapeutic equivalents or later approved drugs which are designated as AB rated by U.S. FDA as per Approved Drug Products with Therapeutic Equivalence Evaluations (34'h edition) or drugs obtained marketing approval by U.S. FDA through Abbreviated New Drug Application (ANDA) filing by establishing bioequivalence to such Product”. For example, TEMOVATE® Cream comprises clobetasol propionate 0.5 mg/g in a cream base of propylene glycol, glyceryl monostearate, cetosteryl alcohol, glyceryl stearate, PEG 100 stearate, white wax, chlorocresol, sodium citrate, citric acid monohydrate, and purified water. TEMOVATE® Ointment comprises clobetasol propionate 0.5 mg/g in a base of propylene glycol, sorbitan sesquioleate, and white petrolatum. Excipient details of other compositions such Therapeutic equivalents/Pharmaceutical equivalents of TEMOVATE® gel or TEMOVATE® gel or TEMOVATE® solution can be found from U.S. FDA or any other public literature. In some embodiments, TEMOVATE® includes its U.S. FDA therapeutic or pharmaceutical equivalents. In some embodiments, TEMOVATE® cream includes its U.S. FDA therapeutic or pharmaceutical equivalents. TEMOVATE® is a Trademark originally registered by Glaxo Group Limited Corporation. The last listed owner of this Trademark is Fougera Pharmaceuticals, Inc. Corporation. TEMOVATE® E is indicated for the relief of the inflammatory and pruritic manifestations of corticosteroid-responsive dermatoses in patients 12 years of age or older and the treatment of moderate to severe plaque-type psoriasis in patients 16 years of age and older. Treatment beyond 4 consecutive weeks and use in pediatric patients less than 16 years of age is not recommended. Safety and effectiveness of Temovate® E in pediatric patients have not been established and its use in pediatric patients under 12 years of age is not recommended. Four-week HPA axis suppression studies with Temovate® E Cream in pediatric subjects have not been conducted. Because of a higher ratio of skin surface area to body mass, pediatric patients are at a greater risk than adults of HPA axis suppression and Cushing's syndrome when they are treated with topical corticosteroids. They are therefore also at greater risk of glucocorticosteroid insufficiency during or after withdrawal of treatment. Adverse effects including striae have been reported with inappropriate use of topical corticosteroids in infants and children. HPA axis suppression, Cushing's syndrome, linear growth retardation, delayed weight gain, and intracranial hypertension have been reported in children receiving topical corticosteroids. Manifestations of adrenal suppression in children include low plasma cortisol levels and absence of response to ACTH stimulation. Manifestations of intracranial hypertension include bulging fontanelles, headaches, and bilateral papilledema. The use of Temovate® E for 4 consecutive weeks has not been studied in pediatric patients under 16 years of age.

### Definitions

The terms as used herein have the following meanings:

Terms such as “about,” “up to”, “generally”, “substantially” and the like are to be construed as modifying a term or value such that it is not an absolute. Such terms will be defined by the circumstances and the terms that they modify as those terms are understood by those of skill in the art. This includes, at the very least, the degree of expected experimental error, technical error and instrumental error for a given experiment, technique or an instrument used to measure a value.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the present invention.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The present invention can comprise or consist essentially of the components of the present invention as well as other ingredients or elements described herein. As used herein, “comprising” means the elements recited, or their equivalent in structure or function, plus any other element or elements which are not recited. The terms “having,” “including,” and “comprised of are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention.

As used herein, “optional” or “optionally” means that the subsequently described event or circumstance does or does not occur or exist and that the description includes instances where said event or circumstance occurs or exists, and instances where it does not.

As used herein, the terms “treatment” or “treating” relate to curing or substantially curing a condition, as well as ameliorating at least one symptom of the condition, and are inclusive of prophylactic treatment and therapeutic treatment. As would be recognized by one or ordinary skill in the art, treatment that is administered prior to clinical manifestation of a condition then the treatment is prophylactic (i.e., it protects the subject against developing the condition). If the treatment is administered after manifestation of the condition, the treatment is therapeutic (i.e., it is intended to diminish, ameliorate, control, or maintain the existing condition and/or side effects associated with the condition). The terms relate to medical management of a subject with the intent to substantially cure, ameliorate, stabilize, or substantially prevent a condition, including but not limited to prophylactic treatment to preclude, avert, obviate, forestall, stop, or hinder something from happening, or reduce the severity of something happening, especially by advance action. As such, the terms treatment or treating include, but are not limited to: inhibiting the progression of a condition of interest; arresting or preventing the development of a condition of interest; reducing the severity of a condition of interest; ameliorating or relieving symptoms associated with a condition of interest; causing a regression of the condition of interest or one or more of the symptoms associated with the condition of interest; and preventing a condition of interest or the development of a condition of interest.

The term “localized region,” as used herein refers to a discrete location on the body surface of the subject, such as a location experiencing a symptom of condition being treated.

“Clinically significant” means a change that will produce an physiological effect.

The term “post treatment” as used herein to refer to the time period post to the topical treatment course of about 2 weeks or 15 days.

The term “improved efficacy” or “improving efficacy” or “improving therapeutic efficacy” as used herein refers to the therapeutically beneficial effects of the topical active with reduction of systemic adverse effects as described in the present invention.

The term “therapeutic efficacy” as used herein means a change in the severity of a subject's condition from “very severe or severe” or “moderate conditions” to “mild” or “minimal or almost clear” or “clear” lesions after the scheduled treatment period or an improvement in the subjects' Investigator Global Assessment (IGA) score.

The term “subject” as used herein refers to a patient suffering from skin disorders such as psoriasis. As used herein, the term “subject” includes both human and animal subjects. Thus, veterinary therapeutic uses, as well as uses in connection with human subjects, are provided in accordance with the present invention. The terms “pediatric subject” or “pediatric patient” are used herein to indicate a subject that is a male or female aged about 6 to about 16 years and 11 months of age, or a male or female aged about 12 to about 16 years and 11 months of age, or a male or female under the age of 18 years, or a male or female under the age of 12 years.

The term “adverse effect” as used herein means adverse effects of the high-mid potent topical steroids such as clobetasol, and the adverse effects are significant effect on endocrine system. Adverse effects as defined in this application encompass reversible suppression of the hypothalamic-pituitary-adrenal (HPA) axis.

The term “substantially free of adverse effects” as used herein indicates that administration of the composition to the pediatric subject results in at least a 70% decrease in adverse effects. In some embodiments, “substantially free of adverse effects” as used herein indicates that administration of the composition to a pediatric population results in at least about 60% of the total patient population that does not have adverse effects. In some embodiments, “substantially free of adverse effects” as used herein indicates that administration of the composition to an individual pediatric patient results in at least about 60% chance that the patient will not develop adverse effects.

The term “substantially free” as used herein indicates that the specified substance referred to is present in amounts not more than 10% by weight of the total composition. In other embodiments, the term “substantially free” as used herein indicates that plasma concentrations of clobetasol that are insufficient to reduce serum cortisol levels less than or equal to 18 μg/dL.

The term “clobetasol”, as used herein, encompasses pharmaceutically acceptable, pharmacologically active derivatives of clobetasol, including clobetasol propionate, clobetasol base form, its ester form, its isomer form, both individual enantiomers of clobetasol (dextrogyral and levogyral enantiomers) in their substantially pure form and their pharmaceutically acceptable salts, mixtures (in any ratio) of clobetasol enantiomers and their pharmaceutically acceptable salts, and active metabolites of clobetasol and their pharmaceutically acceptable salts, unless otherwise noted. The solid state form of clobetasol used in the composition of the present invention is not critical. For example, clobetasol propionate can be amorphous or crystalline. As will be recognized by one of ordinary skill in the art upon study of this application, clobetasol(s) are corticosteroids. In some embodiment, the terms “active”, “active agent”, or “compound” herein refers to corticosteroids, including clobetasol, or to pharmaceutically acceptable forms thereof.

The term “low-dose clobetasol” means clobetasol is present in an amount from about 0.005% to about 0.045% (w/w).

The term “clobetasol plasma levels insufficient to reduce serum levels cortisol less than or equal to 18 μg/dL” is used herein to indicate any plasma concentration of clobetasol which does not provide HPA axis suppression to the subject treated with topical composition of the present invention, and such plasma concentrations may be selected from about 1000 pg/ml to about 10 pg/ml or below quantifiable limit (<=10 pg/ml).

The term “highest approved topical dose of clobetasol” as used herein refers to a highest approved topical dose of clobetasol propionate by U.S. Food and drug administration (U.S. FDA) for the treatment of one or more of skin disorders and the highest approved topical dose of clobetasol propionate by US FDA is 0.05% (w/w), i.e. TEMOVATE® or TEMOVATE® E. The term “TEMOVATE®” or TEMOVATE® E is used interchangeably for indicating “highest approved topical dose of clobetasol propionate’ i.e. 0.05% (w/w) in cream or gel or ointment or solution form; or its pharmaceutical equivalents or its therapeutic equivalents or later approved drugs which are designated as AB rated by U.S. FDA as per Approved Drug Products with Therapeutic Equivalence Evaluations (34'h edition) or drugs obtained marketing approval by U.S. FDA through Abbreviated New Drug Application (ANDA) filing by establishing bioequivalence to such Product”.

The term “plasma concentrations of clobetasol” as used herein indicates that plasma concentrations of clobetasol base or its pharmaceutically acceptable salts or degradants, unless until specific salt form is denoted; or in some embodiments, “plasma concentrations of clobetasol” indicates plasma concentrations of clobetasol propionate or clobetasol base.

The term ‘carrier’ or “vehicle” denotes organic or inorganic ingredients, natural or synthetic, with which an active ingredient is combined to facilitate application of a composition. Examples of carriers include, but not limited to, water, acetone, alone or in combination with materials such as silicone fluids. In certain embodiments, the carrier can comprise, in addition to water, water-immiscible substances such as any pharmaceutically acceptable fatty esters of natural fatty acids, triglycerides of animal or vegetable, medium chain triglycerides, mixtures of mono-, di- and/or triglycerides, waxes, hydrogenated vegetable oils, and mixtures thereof.

The term “aqueous-based” is defined as an emulsion which comprises high percentage of water.

The term “pharmaceutically-acceptable” as used herein, means that inert excipients are suitable for use in contact with the tissues of humans and lower animals without undue toxicity, incompatibility, instability, irritation, allergic response, and the like, commensurate with a reasonable benefit/risk ratio.

The term “penetration enhancing agent(s)” as used herein means compounds that enhance the penetration rate of a corticosteroid through the skin or mucous membrane, such as by temporarily diminishing the impermeability of the skin or mucous membrane. Generally, a penetration enhancing agent is a component used to enhance the penetration rate of steroid through the skin or mucous membrane, such as by temporarily diminishing the impermeability of the skin or membrane. Penetration enhancing agents have also been known as “accelerants” and “absorption promoters.” Examples of suitable penetration enhancing agents include, but are not limited to, polyols, glycols (except propylene glycol), ethers, glycol ethers, esters, sulfoxides, fatty acids, fatty acid esters, fatty alcohols, essential oils, terpenes, terpenoids, PEGylated fatty acids, PEGylated fatty acid esters, PEGylated fatty alcohols and mixtures thereof, including polyethylene glycol, polyethylene glycol monolaurate, and butanediol; sulfoxides, including dimethylsulfoxide and decylmethylsulfoxide; ethers, including diethylene glycol monoethyl ether and diethylene glycol monomethyl ether; fatty acids, including lauric acid, oleic acid, and valeric acid; fatty acid esters, including isopropyl myristate, isopropyl palmitate, methyl propionate, and ethyl oleate; nitrogenous compounds including urea, dimethyl acetamide, dimethylformamide 2-pyrrolidone, ethanolamine, methyl-2-pyrrolidone, diethanolamine, and triethanolamine; terpenes; terpenoids; alkanones; organic acids, including salicylic acid, citric acid, and succinic acid; and combinations comprising one or more of the foregoing materials. In some embodiments, the penetration enhancing agent used in the pharmaceutical composition of the present invention is diethylene glycol monoethyl ether. In some embodiments, the penetration enhancing agent is not polypropylene glycol. The penetration enhancing agent(s) may interchangeably be used as solvent.

The term “enhanced flux” as used herein refers to increase in the skin permeation of the active in skin layers of the subject up to dermis with less systemic exposure, i.e., enhanced flux allows for the use of a lower dose of active to treat disease condition effectively.

The term “emollients” are substances that soften and soothe the skin. They are used to prevent dryness and scaling of the skin. Examples of emollients that can be used in the present invention include, but not limited to, oils of natural origin such as almond oil, coconut oil, olive oil, palm oil, peanut oil and the like, fatty acids such as lauric acid, myristic acid, palmitic acid, and stearic acid, monohydric alcohol esters of the fatty acids such as ethyl laurate, isopropyl laurate, ethyl myristate, n-propyl myristate, isopropyl myristate, ethyl palmitate, isopropyl palmitate, methyl palmitate, methyl stearate, ethyl stearate, isopropyl stearate, butyl stearate, isobutyl stearate, amyl stearate, and isoamyl stearate, glycols such as ethylene glycol, diethylene glycol, polyethylene glycol, branched aliphatic alcohols such as lauryl alcohol, myristyl alcohol, and stearyl alcohol, or mixtures thereof. Exemplary emollients include caprylic/capric triglyerides, castor oil, ceteareth-20, ceteareth-30, cetearyl alcohol, ceteth 20, cetosteryl alcohol, cetyl alcohol, cetyl stearyl alcohol, cocoa butter, diisopropyl adipate, glycerin, glyceryl monooleate, glyceryl monostearate, glyceryl stearate, isopropyl myristate, isopropyl palmitate, lanolin, lanolin alcohol, hydrogenated lanolin, liquid paraffins, linoleic acid, mineral oil, oleic acid, white petrolatum, polyethylene glycol, polyoxyethylene glycol fatty alcohol ethers, silicones and mixtures thereof. Silicones are typically organically modified organopoly siloxanes, sometimes called silicone surfactants. Useful polysiloxane or silicone emollients include, but not limited to, polysiloxane polymer, dimethicone copolyols, cyclomethicones. These materials are polydimethyl siloxanes, which have been modified to include polyether side chains such as polyethylene oxide chains, polypropylene oxide chains, mixtures of these chains, and polyether chains containing moieties derived from both ethylene oxide and propylene oxide.

The term “antioxidants” are substances which inhibit oxidation or suppress reactions promoted by oxygen or peroxides. Antioxidants, especially lipid-soluble antioxidants, can be absorbed into the cellular membrane to neutralize oxygen radicals and thereby protect the membrane. Suitable antioxidants that can be used in the present invention include, but not limited to, ascorbic acid (vitamin C), glutathione, lipoic acid, uric acid, sorbic acid, carotenes, a-tocopherol (vitamin E), TPGS, ubiquinol, butylated hydroxyanisole, butylated hydroxytoluene, sodium benzoate, propyl gallate (PG, E310), and tertiary-butylhydroquinone.

The term “preservative” refers to a natural or synthetic chemical that prevents the decomposition of the composition by microbial growth or by undesirable chemical changes. Preservatives can desirably be incorporated into a composition for protecting against the growth of potentially harmful microorganisms. While microorganisms tend to grow in an aqueous phase and can also reside in a hydrophobic or oil phase. Examples of preservatives that can be used in the present invention include, but not limited to, methylparaben, propylparaben, benzyl alcohol, chlorocresol, benzalkonium chloride, cetrimonium chloride, sodium edetate, boric acid, sorbic acid, or any mixtures thereof.

The term “thickening agents” or “gelling agents” are used to give bulkiness to the composition. Examples of thickening agents or gelling agents that can be used in the present invention include, but not limited to carbomers, polyethylene glycols, acrylate polymers, methacrylate polymers, polyvinylpyrrolidones, copolymers based on butyl methacrylate and methyl methacrylate povidone, vinyl acetates, polyvinyl acetates, celluloses, gums, alginates, cellulose acetate phthalates, cellulose acetate butyrates, hydroxypropyl methyl cellulose phthalates, and the like. Examples include CARBOPOL® products, PEG 400, EUDRAGIT® 100, EUDRAGIT® RSPO, EUDRAGIT® RLPO, EUDRAGIT® ND40, PLASDONE®, copolymers based on butyl methacrylate and methyl methacrylate (PLASTOID® B), alkyl celluloses such as ethyl celluloses and methyl celluloses, hydroxyalkyl celluloses such as hydroxyethyl cellulose and hydroxypropyl cellulose, hydroxyalkyl alkyl celluloses such as hydroxypropyl methyl celluloses and hydroxybutyl methyl celluloses, gums such as xanthan gum, tragacanth, guar gum, locust bean gum, acacia, and the like. In an embodiment, the thickening agents are non-polymeric thickening agents, examples of non-polymeric thickening agent are fatty alcohol selected from group comprising: cetyl alcohol, paraffin, stearyl alcohol, white wax, wax cetyl esters, microcrystalline wax, anionic emulsifying wax, nonionic emulsifying wax, yellow wax, castor oil, ceresin, cetosteryl alcohol, cyclomethicone, glyceryl behenate, hectorite, myristyl alcohol, cetylstearyl alcohol, triolein, and lanolin. Fatty alcohols that can be used as non-polymeric thickening agent, include but not limited to stearyl alcohol, oleyl alcohol, cetyl alcohol, cetosteryl alcohol are long chain fatty alcohols. Stearyl Alcohol is a white, waxy solid with a faint odor, while oleyl alcohol and octyl dodecanol are clear, colorless liquids. Oleyl alcohol is an unsaturated fatty alcohol, similar to the saturated fatty alcohols stearyl alcohol and cetyl alcohol. In an embodiment, the topical compositions of the present invention are substantially free of polymers.

Other thickening agents or gelling agents or polymers that are useful in the present invention include, but not limited to, polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyglycolides, polysiloxanes, polyurethanes and copolymers thereof, cellulose ethers, cellulose esters, nitrocellulose's, polymers of acrylic and methacrylic esters, cellulose acetates, cellulose propionates, cellulose acetate butyrates, cellulose acetate phthalates, carboxylethyl celluloses, cellulose triacetates, cellulose sulphate sodium salts, poly(methyl ethacrylate), poly(ethylmethacrylate), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylenes, polypropylenes, poly(ethylene glycol), poly(ethylene oxide), poly(ethylene terephthalate), poly(vinyl alcohol), poly(vinyl acetate), poly (vinyl chloride), polystyrenes, and the like, including their mixtures thereof.

Examples of other useful polymers that can act as thickening agents or gelling agents include, but not limited to, synthetic polymers, such as polymers of lactic acid and glycolic acid, polyanhydrides, poly(ortho ester), polyurethanes, poly(butyric acid), poly(valeric acid), poly(caprolactone), poly(hydroxybutyrate), poly(lactide-co-glycolide), poly(lactide-co-caprolactone), and natural polymers such as alginate and other polysaccharides that include but not limited to arabinans, fructans, fucans, galactans, galacturonans, glucans, mannans, xylans (such as, for example, inulin), levan, fucoidan, carrageenan, galactocarolose, pectic acid, pectin, amylase, pullulan, glycogen, amylopectin, cellulose, dextran, pustulan, chitin, agarose, keratan, chondroitan, dermatan, hyaluronic acid, alginic acid, xanthan gum, starches, and various other natural homopolymers and heteropolymers, such as those containing one or more of aldoses, ketoses, acids or amines, erythrose, threose, ribose, arabinose, xylose, lyxose, allose, altrose, glucose, mannose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof, and including dextran and cellulose, collagen, albumin and other hydrophilic proteins, zein and other prolamines and hydrophobic proteins, copolymers and mixtures thereof.

The term “humectant” refers to a hygroscopic substance that is often a molecule with several hydrophilic groups, most often hydroxyl groups, but amines and carboxyl groups, sometimes esterified, can be encountered as well; the affinity to form hydrogen bonds with molecules of water is crucial here. Examples of humectants include, but not limited to, glycerol, and glyceryl triacetate (E1518). Others can be sugar polyols like sorbitol (E420), xylitol and maltitol (E965), polymeric polyols like polydextrose (E1200), or natural extracts like quillaia (E999), lactic acid or urea.

The term “solvent” refers to components that aid in the dissolution of the drug in the formulation. Solvents serve to maintain a solution of the drug in the composition. Some solvents can also enhance percutaneous penetration of drug and/or act as humectants. For topical corticosteroids, solvents can include water-immiscible substances such as fatty esters of natural fatty acids, triglycerides of animal or vegetable, medium chain triglycerides, mixtures of mono-, di- and/or triglycerides, waxes, hydrogenated vegetable oils, and mixtures thereof. Some specific examples include, but not limited to, castor oil, isopropyl myristate, dimethyl isosorbide, oleyl alcohol, labrafil, labrasol, medium chain triglyceride, diethyl sebacate, lanolin oil, citrate triisocetyl triglycerides having 10-18 carbon atoms, caprylic/capric triglycerides, coconut oil, corn oil, cottonseed oil, linseed oil, oil of mink, olive oil, palm oil, sunflower oil, nut oil, saturated paraffin oils, mineral oils, vegetable oils or glycerides, and the like. Solvent can also be selected from the group comprising monoalkyl ether of diethylene glycol such as diethylene glycol monomethyl ether, diethylene glycol monoethyl ether or mixtures thereof. In some embodiments, the solvent is diethylene glycol monoethyl ether. It is marketed by Gattefosse under the trade name TRANSCUTOL®, TRANSCUTOL-P®, TRANSCUTOL-CG®, and TRANSCUTOL-HP®.

Some of the excipients described above can have more than one function in a composition. For example, an excipient can be both a solvent and a penetration enhancer, or both a solvent and a carrier. The categorizations of excipients described above are not to be construed as limiting or restricting in any manner.

Described herein are methods comprising topically administering twice daily for up to about two weeks to a pediatric subject a composition comprising about 0.05% to about 0.045% (w/w) of clobetasol, an aqueous phase, an oil phase; about 5% of an emollient, about 0.05% of an antioxidant, about 10% of a solvent, about 6% of an emulsifier, and a preservative; wherein the aqueous phase is water and the water is at least 60% the total weight of the composition, wherein the oil phase comprises at least one penetration enhancing agent in an amount from about 0.01% to about 15.0% of the total weight of the composition and a non-polymeric thickening agent, wherein the pediatric subject has a risk of HPA axis suppression that is similar to the risk of HPA axis suppression in an adult subject.

In some embodiments, the composition is topically administered to a pediatric subject with moderate to severe plaque psoriasis. In some embodiments, the pediatric subject has moderate to severe psoriatic lesions involving at least about 10% body surface area. In some embodiments, the pediatric subject has an investigator global assessment score of greater than or equal to 3. In some embodiments, treating moderate to severe plaque psoriasis in the subject results in an investigator global assessment score of 0 to 1. In some embodiments, the investigator global assessment score is measured at day 15.

In some embodiments, the pediatric subject is a male or female aged about 6 to about 16 years and 11 months of age. In some embodiments, the pediatric subject is a male or female aged about 12 to about 16 years and 11 months of age. In some embodiments, the pediatric subject is a male or female under the age of 18 years. In some embodiments, the pediatric subject is a male or female under the age of 12 years.

In some embodiments, topical administration of the composition to a pediatric subject results in an at least 80% chance that the subject will not develop hypothalamic-pituitary-adrenal (HPA) axis suppression. In some embodiments, topical administration of the composition to a pediatric subject results in plasma concentrations of clobetasol that are insufficient to reduce serum cortisol levels less than or equal to 18 μg/dL. In some embodiments, topical administration of the composition to a pediatric subject results in the subject being substantially free of adverse effects. In further embodiments the adverse effect is selected from skin irritation, vein collapse, itching, burning, stinging and a combination thereof. In further embodiments the adverse effect is selected from Cushing's syndrome, hyperglycemia and glycosuria, linear growth retardation, delayed weight gain, and intracranial hypertension and a combination thereof. In some embodiments, topical administration to a pediatric subject results in no HPA axis suppression. In some embodiments, topical administration in a pediatric subject results in substantially no HPA axis suppression.

In further embodiments, the compositions of the present invention using one or more other corticosteroids can be prepared by using a process similar to that described above. The topical pharmaceutical composition of the present invention is useful in the prophylaxis, amelioration or treatment of skin diseases or disorders such as psoriasis/psoriatic plaques, relief of the inflammatory and pruritic manifestations of steroid responsive dermatoses, erythema, contact sensitivity reactions, atopic dermatitis, seborrheic dermatitis, eczema, plaque psoriasis, erythrodermic psoriasis, psoriasis of the scalp, and other associated diseases or disorders in pediatric subjects. In some embodiments, of the present invention, it was surprisingly found that the topical compositions of the invention containing an oil phase that comprises at least one penetration enhancing agent, and an aqueous phase, provides an enhanced flux of clobetasol through the localized region of the body surface to reach the dermis layer; this advantageously allows for the use of a lower concentration of clobetasol, while providing no significant effect on the endocrine system i.e., HPA axis suppression in pediatric subjects. In some embodiments, of the invention, the pharmaceutical compositions of the present invention may contain 3% of a penetration enhancing agent. In further embodiments of the invention, the pharmaceutical compositions of the present invention may contain 10% of a penetration enhancing agent. In some embodiments, administration of the topical compositions results in HPA axis suppression in less than about 15% of the pediatric subjects treated. In some embodiments, administration of the topical compositions results in HPA axis suppression without any clinical symptoms.

In some embodiments, the clobetasol is clobetasol proprionate. In some embodiments, the topical administration of the composition provides a mean clobetasol plasma level less than about 10 pg/mL.

In some embodiments, the emollient comprises cyclomethicone. In some embodiments, the antioxidant comprises butylated hydroxytoluene. In some embodiments, the solvent comprises isopropyl myristate. In some embodiments, the emulsifier comprises glyceryl stearate and PEG 100 stearate. In some embodiments, the preservative comprises one or both of methylparaben and propylparaben. In further embodiments, the preservative is methylparaben and the methylparaben is about 0.2% of the total weight of the composition. In further embodiments, the preservative is propylparaben and propylparaben is about 0.4% of the total weight of the composition. In some embodiments, the penetration enhancing agent is diethylene glycol monoethyl ether. In some embodiments, the non-polymeric thickening agent comprises one or both of cetosteryl alcohol and white wax. In some embodiments, the oil phase comprises at least one penetration enhancing agent in an amount up to about 5.0% of the total weight of the composition, wherein the at least one penetration enhancing agent is diethylene glycol monoethyl ether. In some embodiments, the oil phase comprises at least one penetration enhancing agent in an amount of about 3.0% of the total weight of the composition, wherein the at least one penetration enhancing agent is diethylene glycol monoethyl ether.

In some embodiments, the topically administered composition is substantially free of propylene glycol.

In some embodiments, the topically administered composition is substantially free of polymers.

In some embodiments, the topical compositions of the present invention comprise a therapeutically effective amount of clobetasol; an oil phase comprising at least one skin penetration enhancer; an aqueous phase and optionally one pharmaceutically acceptable excipient.

In some embodiments, the topical composition provides a mean clobetasol plasma level less than about 10 pg/mL. In some embodiments, the clobetasol is clobetasol propionate.

In some embodiments, the non-polymeric thickening agent is cetosteryl alcohol.

In some embodiments, the at least one pharmaceutically acceptable excipient is selected from the group consisting of a carrier, emulsifier, co-emulsifier, solvent, co-solvents, emollient, antioxidant, preservative, gelling or thickening agent, polymer, surfactant, soothing agent, pH modifier, solubilizer, humectants, moisturizer, oily base, and any combination thereof. In some embodiments, the at least one pharmaceutically acceptable excipient is an emulsifier and the emulsifier is a mixture of glyceryl stearate and PEG 100 stearate. In some embodiments, the mixture of glyceryl stearate and PEG 100 Stearate is about 6% of the total weight of the composition. In some embodiments, the isopropyl myristate is about 10% of the total weight of the composition. In some embodiments, the at least one pharmaceutically acceptable excipient is an emollient and the emollient is cyclomethicone. In some embodiments, the cyclomethicone is about 5% of the total weight of the composition. In some embodiments, the at least one pharmaceutically acceptable excipient is an antioxidant and the antioxidant is butylated hydroxytoluene. In some embodiments, the butylated hydroxytoluene is about 0.05% of the total weight of the composition. In some embodiments, the at least one pharmaceutically acceptable excipient is a preservative and the antioxidant is selected from methylparaben, propylparaben and a combination thereof. In some embodiments, the preservative is methylparaben and the methylparaben is about 0.2% of the total weight of the composition. In some embodiments, the preservative is propylparaben and the propylparaben is about 0.4% of the total weight of the composition. In some embodiments, the at least one pharmaceutically acceptable excipient is a gelling or thickening agent and the gelling or thickening agent is white wax. In some embodiments, the white wax is about 1% of the total weight of the composition. In some embodiments, the aqueous phase is water. In some embodiments, the water is at least 60% of the total weight of the composition. In some embodiments, the topical composition is substantially free of propylene glycol. In some embodiments, the topical composition is substantially free of polymers. In some embodiments, the oil phase comprises at least one penetration enhancing agent in an amount from about 0.01% to about 10.0% of the total weight of the composition, wherein the at least one penetration enhancing agent is diethylene glycol monoethyl ether. In some embodiments, the oil phase comprises at least one penetration enhancing agent in an amount up to about 5.0% of the total weight of the composition, wherein the at least one penetration enhancing agent is diethylene glycol monoethyl ether. In some embodiments, the oil phase comprises at least one penetration enhancing agent in an amount of about 3.0% of the total weight of the composition, wherein the at least one penetration enhancing agent is diethylene glycol monoethyl ether.

In some embodiments, the topically administered composition comprises at least one pharmaceutically acceptable excipient. In some embodiments, the at least one pharmaceutically acceptable excipient is selected from the group consisting of a carrier, emulsifier, co-emulsifier, solvent, co-solvents, emollient, antioxidant, preservative, gelling or thickening agent, polymer, surfactant, soothing agent, pH modifier, solubilizer, humectants, moisturizer, oily base, and any combination thereof. In some embodiments, the at least one pharmaceutically acceptable excipient is an emollient and the emollient is cyclomethicone. In some embodiments, the cyclomethicone is about 5% of the total weight of the composition. In some embodiments, the at least one pharmaceutically acceptable excipient is an antioxidant and the antioxidant is butylated hydroxytoluene. In some embodiments, the butylated hydroxytoluene is about 0.05% of the total weight of the composition. In some embodiments, the at least one pharmaceutically acceptable carrier is a solvent and solvent is isopropyl myristate. In some embodiments, the isopropyl myristate is about 10% of the total weight of the composition. In some embodiments, the at least one pharmaceutically acceptable excipient is an emulsifier and the emulsifier is a mixture of glyceryl stearate and PEG 100 stearate. In some embodiments, the mixture of glyceryl stearate and PEG 100 Stearate is about 6% of the total weight of the composition. In some embodiments, the at least one pharmaceutically acceptable excipient is a preservative and the preservative is selected from methylparaben, propylparaben and a combination thereof. In some embodiments, the preservative is methylparaben and the methylparaben is about 0.2% of the total weight of the composition. In some embodiments, the preservative is propylparaben and the propylparaben is about 0.4% of the total weight of the composition. In some embodiments, the at least one pharmaceutically acceptable excipient is a gelling or thickening agent and the gelling or thickening agent is white wax. In some embodiments, the white wax is about 1% of the total weight of the composition. In some embodiments, the aqueous phase is water. In some embodiments, the water is at least 60% of the total weight of the composition. In some embodiments, the topical composition is substantially free of propylene glycol. In some embodiments, the topical composition is substantially free of polymers. In some embodiments, the oil phase comprises at least one penetration enhancing agent in an amount from about 0.01% to about 10.0% of the total weight of the composition, wherein the at least one penetration enhancing agent is diethylene glycol monoethyl ether. In some embodiments, the oil phase comprises at least one penetration enhancing agent in an amount up to about 5.0% of the total weight of the composition, wherein the at least one penetration enhancing agent is diethylene glycol monoethyl ether. In some embodiments, the oil phase comprises at least one penetration enhancing agent in an amount of about 3.0% of the total weight of the composition, wherein the at least one penetration enhancing agent is diethylene glycol monoethyl ether.

In some embodiments, the topical composition comprises about 0.025% clobetasol; an oil phase of about 3% diethylene glycol monoethyl ether as the penetration enhancing agent and about 1% white wax as the non-polymeric thickening agent; about 10% isopropyl myristate; water as the aqueous phase; and wherein the at least one pharmaceutically acceptable excipient is about 6% of a mixture of glyceryl stearate and PEG 100 stearate, about 0.05% butylated hydroxytoluene, about 5% cyclomethicone, about 0.2% methylparaben; and about 0.4% propylparaben.

In some embodiments, the topical composition comprises about 0.025% clobetasol; an oil phase of about 3% diethylene glycol monoethyl ether as the penetration enhancing agent and about 1% white wax as the non-polymeric thickening agent; about 10% isopropyl myristate; water as the aqueous phase; and wherein the at least one pharmaceutically acceptable excipient is about 6% of a mixture of glyceryl stearate and PEG 100 stearate, about 0.05% butylated hydroxytoluene, about 5% cyclomethicone, about 0.2% methylparaben; and about 0.4% propylparaben.

In some embodiments, the topical pharmaceutical compositions comprise at least one corticosteroid and at least one penetration enhancing agent. In some embodiments, the compositions of the present invention are substantially free of propylene glycol. In some embodiments, the compositions include not more than 10, 9, 8, 7, 6, 5, 4, 3, 2, or 1% propylene glycol by weight of the total composition. Thus, in some embodiments, the composition is substantially free of polypropylene glycol where there is less than 1% by weight polypropylene glycol in the total composition. In other embodiments, the composition is substantially free of polypropylene glycol where there are less than 2, 3, 4, 5, 6, 7, 8, 9, or 10% by weight polypropylene glycol in the total composition. In other embodiments, the composition is substantially free of polypropylene glycol where there is less than about 0% by weight polypropylene in the total composition.

In some embodiments, the present invention provides for topical pharmaceutical compositions comprising a therapeutically effective amount of clobetasol propionate, at least one penetration enhancing agent and at least one pharmaceutically acceptable excipient, wherein the composition is substantially free of propylene glycol. In some embodiments, the present invention provides a method to provide an enhanced flux of clobetasol propionate through the localized region of the body surface to reach the dermis layer, comprising administering to a subject the effective amount of topical pharmaceutical composition comprising: (a) low dose of clobetasol propionate, (b) an oil phase comprising: at least one penetration enhancing agent and a non-polymeric thickening agent, (c) an aqueous phase; and (d) optionally, at least one pharmaceutically acceptable excipient; wherein the composition is substantially free of propylene glycol and substantially free of polymers.

In some embodiments, the clobetasol present in the composition amounts from about 0.005% to about 0.1% of the total weight of the composition. In some embodiments, the clobetasol propionate is present in amounts from about 0.005% to about 0.05% of the total weight of the composition, or in amounts up to about 0.025% of the total weight of the composition. In some embodiments, low-dose clobetasol is provided from about 0.005% to about 0.045% (w/w). In some embodiments, low-dose clobetasol is provided at a dose of about 0.005%, 0.01%, 0.015%, 0.02%, or 0.025% to about 0.03%, 0.035%, or 0.04% (w/w). In some embodiments, low-dose clobetasol is provided at a dose of about 0.005%, 0.01%, 0.015%, 0.02%, or 0.025% to about 0.03%, 0.035%, or 0.04% (w/w) 0.045% (w/w).

In some embodiments, of the present invention, the clobetasol propionate is present in amounts up to about 0.005, 0.006, 0.007, 0.008, 0.009, 0.010, 0.011, 0.012, 0.013, 0.014, 0.015, 0.016, 0.017, 0.018, 0.019, 0.020, 0.021, 0.022, 0.023, 0.024, 0.025, 0.026, 0.027, 0.028, 0.029, 0.030, 0.031, 0.032, 0.033, 0.034, 0.035, 0.036, 0.037, 0.038, 0.039, 0.040, 0.041, 0.042, 0.043, 0.044, or 0.045% of the total weight of the composition. In some embodiments, the clobetasol propionate is present in amounts of less than 0.050% of the total weight of the composition. In some embodiments, the clobetasol propionate is present in amounts of about 0.010 to about 0.040% of the total weight of the composition. In some embodiments, the clobetasol propionate is present in amounts of about 0.015 to about 0.035% of the total weight of the composition. In some embodiments, the clobetasol propionate is present in amounts of about 0.020 to about 0.030% of the total weight of the composition. In some embodiments, the clobetasol propionate is present in an amount of about 0.025% of the total weight of the composition.

The term “substantially free” as used herein indicates that the specified substance referred to is present in amounts not more than 10% by weight of the total composition or in amounts not more than about 9% by weight of the total composition, or in amounts not more than about 8% by weight of the total composition, or in amounts not more than about 7% by weight of the total composition, or in amounts not more than about 6% by weight of the total composition, or in amounts not more than about 5% by weight of the total composition, or in amounts not more than about 4% by weight of the total composition, or in amounts not more than about 3% by weight of the total composition, or in amounts not more than about 2% by weight of the total composition or in amounts not more than about 1% by weight of the total composition or in an amount about 0% by weight of the total composition or completely free of specified substance i.e. 0%.

In other embodiments, the composition of the present invention comprises at least one penetration enhancing agent in an amount of from about 1% to about 30.0% of the weight of the composition, or in amounts of from about 0.01% to about 10.0% of the composition. In some embodiments, of the present invention, the at least one penetration enhancing agent is provided in amounts up to about 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10% of the weight of the composition.

In another embodiment, the present invention provides for topical pharmaceutical compositions and their uses comprising: a low dose of clobetasol propionate in an amount selected from about 0.005% to about 0.1% of the total weight of the composition; an oil phase comprising at least one penetration enhancing agent in an amount from about 0.01% to about 15.0% of the total weight of the composition and a non-polymeric thickening agent, an aqueous phase, and optionally, at least one pharmaceutically acceptable excipient, wherein the composition is substantially free of propylene glycol and substantially free of polymers.

In another embodiment, the penetration enhancing agent used in the present invention is selected from the group consisting of polyols, glycols (except propylene glycol), ethers, glycol ethers, esters, sulfoxides, fatty acids, fatty acid esters, fatty alcohols, essential oils, terpenes, terpenoids, PEGylated fatty acids, PEGylated fatty acid esters, PEGy-lated fatty alcohols, and mixtures thereof.

In other embodiments of the present invention, the penetration enhancing agent is diethylene glycol monoethyl ether.

In other embodiments, compositions of the present application present invention are physically and chemically stable.

In some embodiments, the low-dose clobetasol is present in an amount from about 0.005% to about 0.04% of the total weight of the composition.

In some embodiments, the low-dose clobetasol is present in an amount from about 0.005% to about 0.04% of the total weight of the composition.

In some embodiments, the low dose clobetasol is about 10% less than the highest approved topical dose of clobetasol 0.05% (w/w), i.e. about 0.04% (w/w) based on total weight of the composition.

In some embodiments, the low dose clobetasol is about 20% less than the highest approved topical dose of clobetasol 0.05% (w/w), i.e. about 0.04% (w/w) based on total weight of the composition.

In some embodiments, the low dose clobetasol is about 30% less than the highest approved topical dose of clobetasol 0.05% (w/w), i.e. about 0.035% (w/w) based on total weight of the composition.

In some embodiments, the low dose clobetasol is about 40% less than the highest approved topical dose of clobetasol 0.05% (w/w), i.e. about 0.03% (w/w) based on total weight of the composition.

In some embodiments, the low dose clobetasol is about 50% less than the highest approved topical dose of clobetasol 0.05% (w/w), i.e. about 0.025% (w/w) based on total weight of the composition.

In some embodiments, the low dose clobetasol is about 60% less than the highest approved topical dose of clobetasol 0.05% (w/w), i.e. about 0.02% (w/w) based on total weight of the composition.

In some embodiments, the low dose clobetasol is about 70% less than the highest approved topical dose of clobetasol 0.05% (w/w), i.e. about 0.015% (w/w) based on total weight of the composition.

In some embodiments, the low dose clobetasol is about 80% less than the highest approved topical dose of clobetasol 0.05% (w/w), i.e. about 0.01% (w/w) based on total weight of the composition.

In some embodiments, the low dose clobetasol is about 90% less than the highest approved topical dose of clobetasol 0.05% (w/w), i.e. about 0.005% (w/w) based on total weight of the composition.

In a preferred aspect, the low-dose clobetasol is about 50% less than the highest approved topical dose of clobetasol 0.05%, i.e. about 0.025% (w/w) based on total weight of the composition.

In some embodiments, the topical compositions comprises a low dose clobetasol of about 0.025% (w/w) which provides equivalent therapeutic efficacy of highest approved topical dose of clobetasol of 0.05% (w/w) in a pediatric subject.

In some aspects of the present invention, the clobetasol is clobetasol propionate i.e. clobetasol-17-propionate.

In some aspects of the present invention, the clobetasol is clobetasol propionate i.e. clobetasol-17-propionate and the clobetasol propionate concentration is in the amount of about 0.025% (w/w).

In some embodiments, the topical compositions of the present invention comprise a) about 0.025% (w/w) of clobetasol; b) an oil phase comprising at least one penetration enhancing agent, and a non-polymeric thickening agent; and c) an aqueous phase.

In some embodiments, the topical cream compositions of the present invention comprise a) about 0.025% (w/w) of clobetasol propionate; b) an oil phase comprising at least one penetration enhancing agent, and a non-polymeric thickening agent; and c) an aqueous phase.

In some embodiments, the topical compositions of the present invention comprise a) about 0.025% (w/w) of clobetasol; b) an oil phase comprising at least one penetration enhancing agent, and a non-polymeric thickening agent; and c) an aqueous phase; and provide mean clobetasol plasma concentrations less than about 130 pg/mL in a pediatric subject. In some embodiments, the clobetasol is clobetasol propionate.

In some embodiments, the topical compositions of the present invention comprise a) about 0.025% (w/w) of clobetasol; b) an oil phase comprising at least one penetration enhancing agent, and a non-polymeric thickening agent; and c) an aqueous phase; and said composition provides post treatment mean clobetasol plasma levels less than about 150 pg/mL in a pediatric subject. In some embodiments, the clobetasol is clobetasol propionate.

In some embodiments, the topical compositions of the present invention comprise a) about 0.025% (w/w) of clobetasol; b) an oil phase comprising at least one penetration enhancing agent, and a non-polymeric thickening agent; and c) an aqueous phase; wherein said composition provides post treatment mean clobetasol propionate plasma levels from about 130 pg/mL to about 10 pg/ml in a pediatric subject. In some embodiments, the clobetasol is clobetasol propionate.

In some embodiments, the topical compositions of the present invention comprise a) about 0.025% (w/w) of clobetasol; b) an oil-in-water emulsion comprising a dispersed oil phase comprising at least one penetration enhancing agent, and a non-polymeric thickening agent, and a continuous aqueous phase; and c) one or more pharmaceutically acceptable excipients. In some embodiments, the clobetasol is clobetasol propionate.

In some embodiments, the topical compositions of the present invention comprise a) about 0.025% (w/w) of clobetasol; b) an oil-in-water emulsion comprising a dispersed oil phase comprising at least one penetration enhancing agent, and a non-polymeric thickening agent, and a continuous aqueous phase; and c) one or more pharmaceutically acceptable excipients; wherein said composition provides mean clobetasol propionate plasma levels less than about 130 pg/mL in a pediatric subject. In some embodiments, the clobetasol is clobetasol propionate.

In some embodiments, the topical compositions of the present invention comprise a) about 0.025% (w/w) of clobetasol; b) an oil-in-water emulsion comprising a dispersed oil phase comprising at least one penetration enhancing agent, and a non-polymeric thickening agent, and a continuous aqueous phase; and c) one or more pharmaceutically acceptable excipients; wherein said composition provides post treatment mean clobetasol propionate plasma levels less than about 150 pg/mL in a pediatric subject. In some embodiments, the clobetasol is clobetasol propionate.

In some embodiments, the topical compositions of the present invention comprise a) about 0.025% (w/w) of clobetasol propionate; b) an oil-in-water emulsion comprising a dispersed oil phase comprising at least one penetration enhancing agent, and a non-polymeric thickening agent, and a continuous aqueous phase; and c) one or more pharmaceutically acceptable excipients; wherein said composition provides post treatment mean clobetasol propionate plasma levels from about 130 pg/mL to about 10 pg/ml in a pediatric subject. In some embodiments, the clobetasol is clobetasol propionate.

In some embodiments, the compositions of the present invention comprise one or more additional active agents that are useful in the management of psoriasis and associated pathological conditions including synthetic, semi-synthetic, or naturally obtained active agents.

In other embodiments, the topical pharmaceutical compositions of the present invention are useful in the relief of the inflammatory and pruritic manifestations of steroid responsive dermatoses, and further can provide a moisturizing and/or soothing effect at the site of application to the skin in pediatric subjects. The compositions of the application reduce the dryness that accompanies the build-up of skin in psoriatic plaques in pediatric subjects.

In other embodiments, the compositions described herein can be applied directly to the psoriatic lesions or dermatoses and can help reduce inflammation, remove built-up scale, reduce skin turnover, and/or clear affected skin of plaques in pediatric subjects. In some embodiment, the compositions of the present invention can utilize any topical corticosteroids, either alone or in combination of others. Suitable examples of topical corticosteroids include, but not limited to, clobetasol propionate, alclometasone dipropionate, amcinonide, beclomethasone dipropionate, betamethasone benzoate, betamethasone dipropionate, betamethasone sodium phosphate, betamethasone valerate, budesonide, clocortolone pivalate, desonide, desoximetasone, dexamethasone, dexamethasone acetate, dexamethasone nicotinate, dexamethasone propionate, dexamethasone sodium phosphate, dexamethasone valerate, diflorasone diacetate, diflucortolone valerate, fluandrenolide, flumethasone pivalate, fluocinolone acetonide, fluocinonide, fluocortin butyl ester, fluticasone propionate, halcinonide, halobetasol propionate, halometasone monohydrate, hydrocortisone, hydrocortisone sodium phosphate, hydrocortisone sodium succinate, hydrocortisone-17-butyrate-21-propionate, hydrocortisone aceponate, hydrocortisone acetate, hydrocortisone valerate, hydrocortisone butyrate, hydrocortisone probutate, methylprednisolone, methylprednisolone acetate, methylprednisolone aceponate, mometasone furoate, prednisolone, prednisolone sodium phosphate, prednisolone acetate, prednisolone-17-valerate-21-acetate, triamcinolone acetonide, triamcinolone acetate, triamcinolone diacetate, and prednicarbate. Other drug compounds are also useful, and this application further specifically contemplates the use of any combinations of steroid drugs.

In an embodiment, the topical compositions of the present invention may be in the form of compositions comprising two phases: an oil phase and an aqueous phase and compositions of the present invention may be in the form of emulsions, creams, lotions, microemulsions, nanoemulsions, emulgels, liposomes, micelles, reverse micelle, spray and the like. In some embodiments, compositions may be in the form of an emulsion. The emulsion can be in the form of an oil-in-water type of emulsion or a water-in-oil type of emulsion. An aqueous-based emulsion, such as an oil-in-water emulsion, frequently has lower viscosity than other emulsion types and exhibits appreciable storage stability and patient compliance. Generally, oil-in-water emulsions have better skin feel properties, when applied to the skin, as these give sensations similar to an aqueous material.

In some embodiments, the pharmaceutical compositions of the present invention are formulated as emulsions, comprising an oily or hydrophobic phase, an aqueous or hydrophilic phase, and an emulsifier. When the oily phase is dispersed as droplets within an aqueous continuous phase, this is called an “oil-in-water” type of emulsion. When the aqueous phase is dispersed as droplets within an oily continuous phase, this is called a “water-in-oil” type of emulsion.

In some embodiments, a pharmaceutical compositions of the present invention are aqueous-based topical oil-in-water emulsion. The aqueous-based oil-in-water emulsion composition of the present invention comprises at least 60% of water in the final composition, or comprises at least 70% of water in the final composition.

In some embodiments, the aqueous-based topical oil-in water emulsion compositions of the present invention comprise: a therapeutically effective amount of a corticosteroid and at least one pharmaceutically acceptable excipient, wherein the composition is substantially free of propylene glycol and substantially free of polymers.

In some embodiments, the aqueous-based topical oil-in water emulsion compositions of the present invention comprise: (a) a therapeutically acceptable amount of clobetasol (b) a discontinuous oil phase comprising: a solvent and at least one penetration enhancing agent; (c) a continuous aqueous phase; and (d) at least one pharmaceutically acceptable excipient, wherein the composition is substantially free of propylene glycol and substantially free of polymers. In some embodiments, the topical compositions comprise: (a) a therapeutically acceptable amount of clobetasol of about 0.025% (w/w); (b) an oil phase comprising: at least one penetration enhancing agent, and a non-polymeric thickening agent; (c) an aqueous phase; and (d) optionally one pharmaceutically acceptable excipient; wherein said topical composition is substantially free of propylene glycol and substantially free of polymers; wherein the topical composition provides no significant adverse effect on endocrine system.

In further embodiments, aqueous based topical oil-in water emulsion compositions of the present invention have a viscosity in the range of from about 10 cP to about 100000 cP. The viscosities of the aqueous-based emulsion compositions of the present invention may be in the range of about 0.01-100 Pascal second, “Pa·s” (10-1,000,000 cP), or about 0.1 to 100 Pa·s (100-1,000,000 cP) or about 1-50 Pa·s (1000-50,000 cP), or about 0.01-15 Pa·s (10-15,000 centipoise, “cP”), or about 0.02-1.5 Pa·s (20-1,500 cP), or about 0.05-1 Pa·s (50-1,000 cP).

In yet other embodiments, the viscosity of the topical compositions of the present invention is in the range of from about 0.1 cP to about 500 cP when measured by Brookfield viscometer Cap 2000+ with spindle no. 1 at 530 rpm at 25° C.

In another embodiment, pharmaceutical composition of the present invention includes one or more pharmaceutically acceptable excipient, which may act as carrier(s), emulsifier(s), co-emulsifier(s) solvent(s), co-solvents(s), emollient(s), antioxidant(s), preservative(s), gelling or thickening agent(s), polymer(s), surfactant(s), soothing agent(s), pH modifier(s), solubilizer(s), humectants(s), moisturizer(s), oily base(s), and the like.

Examples of emulsifying agents include, but not limited to, disodium cocoampho diacetate, oxyethylenated glyceryl cocoate (7 EO), PEG-20 hexadecenyl succinate, PEG-15 stearyl ether, ricinoleic monoethanolamide monosulfosuccinate salts, oxyethylenated hydrogenated ricinoleic triglyceride containing 60 ethylene oxide units such as the products marketed by BASF under the trademarks CREMOPHOR® RH 60 or CREMOPHOr® RH 40 (polyoxyl 40 hydrogenated castor oil), polymers such as poloxamers, which are block copolymers of ethylene oxide and propylene oxide, and the nonsolid fatty substances at room temperature (that is to say, at temperatures ranging from about 20 to 35° C.) such as sesame oil, sweet almond oil, apricot stone oil, sunflower oil, octoxyglyceryl palmitate (or 2-ethylhexyl glyceryl ether palmitate), octoxyglyceryl behenate (or 2-ethylhexyl glyceryl ether behenate), dioctyl adipate, and tartrates of branched dialcohols. Sorbitan fatty acid esters are a series of mixtures of partial esters of sorbitol and its mono and dianhydrides with fatty acids. Sorbitan esters include products marketed as ARLACEL® 20, ARLACEL 40, ARLACEL 60, ARLACEL 80, ARLACEL 83, ARLACEL 85, ARLACEL 987, ARLACEL C, PEG-6 stearate and glycol stearate and PEG-32 stearate (TEFOSE® 63), and PEG-6 stearate and PEG-32 stearate (TEFOSE® 1500), glyceryl stearate and PEG 100 stearate (TEFOSE® 165) and any mixtures thereof. Polyethylene glycol ethers of stearic acid are in another group of emulsifiers that can be used in the emulsions. Examples of polyethylene glycol ethers of stearic acid include, but not limited to, steareth-2, steareth-4, steareth-6, steareth-7, steareth-10, steareth-11, steareth-13, steareth-15, steareth-20, polyethylene glycol ethers of stearyl alcohol (steareth 21), and any mixtures thereof. Other emulsifying agents include sodium lauryl sulphate, cetyl trialkyl ammonium bromide, polyoxyethylene sorbitan fatty acid esters, and any mixtures thereof

Nonionic emulsifying agents include those that can be broadly defined as condensation products of long chain alcohols, e.g., C8-30 alcohols, with sugar or starch polymers, i.e., glycosides. Various sugars include, but not limited to, glucose, fructose, mannose, and galactose, and various long chain alcohols include, but are not limited to, decyl alcohol, cetyl alcohol, stearyl alcohol, lauryl alcohol, myristyl alcohol, oleyl alcohol, and any mixtures thereof.

Other useful nonionic emulsifying agents include condensation products of alkylene oxides with fatty acids such as alkylene oxide esters of fatty acids. Other nonionic surfactants are the condensation products of alkylene oxides with 2 moles of fatty acids such as alkylene oxide diesters of fatty acids.

Emulsifying agents can also include any of a wide variety of cationic, anionic, zwitterionic, and amphoteric surfactants that are known in the art. Examples of anionic emulsifying agents include, but not limited to, alkyl isethionates, alkyl and alkyl ether sulfates and salts thereof, alkyl and alkenyl ether phosphates and salts thereof, alkyl methyl taurates, and soaps (e.g., alkali metal salts and sodium or potassium salts) of fatty acids.

Examples of amphoteric and zwitterionic emulsifying agents include those which are broadly described as derivatives of aliphatic secondary and tertiary amines in which the aliphatic radical can be straight or branched chain, wherein one of the aliphatic substituents contains from about 8 to about 22 carbon atoms and one contains an anionic water solubilizing group, e.g., carboxy, sulfonate, sulfate, phosphate, or phosphonate. Specific examples include, but not limited to, alkylimino acetates, iminodialkanoates and aminoalkanoates, imidazolinium and ammonium derivatives. Other suitable amphoteric and zwitterionic emulsifying agents include betaines, sultaines, hydroxysultaines, alkyl sarcosinates, and alkanoyl sarcosinates.

Silicone emulsifying agents are typically organically modified organopoly siloxanes, sometimes called silicone surfactants. Useful silicone emulsifying agents include dimethicone copolyols. These materials are polydimethyl siloxanes, which have been modified to include polyether side chains such as polyethylene oxide chains, polypropylene oxide chains, mixtures of these chains, and polyether chains containing moieties derived from both ethylene oxide and propylene oxide.

Co-emulsifiers or secondary emulsifying agents include, but not limited to, polyoxylglycerides such as oleoyl macrogolglycerides (LABRAFIL® M 1944CS), linoleoyl macrogolglycerides (LABRAFIL® M 2125CS), caprylocaproyl macrogolglycerides (LABRASOL®), cetyl alcohol (and) ceteth-20 (and) steareth-20 (EMULCIRE™ 61 WL 2659), glyceryl stearate (and) PEG-75 stearate (GELOT® 64), d-alpha tocopheryl polyethylene glycol 1000 succinate (TPGS) and any mixtures thereof.

In some embodiments, a solvent is selected from the group consisting of: mineral oil, isopropyl myristate, dimethyl isosorbide, oleyl Alcohol, labrafil, labrasol, medium chain triglyceride, diethyl sebacate, ammonium lauryl sulfate, lauramine oxide, sodium laureth sulfate, n-methyl-2-pyrrolidinone, octanoic acid, cocobetaine, dimethylsulfoxide, sodium laureth 2 sulfate, benzyl alcohol, ethylacetate, lactic acid, oleic acid, ethylacetate, spearmint oil, isostearic acid, ethanol, propylene glycol diacetate, dimethyl isosorbide, 1-butanol, methyl gluceth-10, sodium lauroylsarcosinate, polysorbate 20, isopropyl alcohol, 1-butanol, Capryol 90, sorbitanmonooleate, glyceryl ricinoleate, poloxamer, polyethylene glycol 200, polysorbate 65, triacetin, benzylalcohol, castor oil, arlacel 165, propylene glycol ricinoleate, glyceryl isostearate, propylene glycol, diethyl phthalate, glyceryl oleate, PEG-8 laurate, sorbitan sesquioleate, PPG26 oleate, 1-octanol, Lauroglycol FCC, diisopropyladipate, laureth 4, and diethyl sebacate for solubilizing clobetasol propionate. The compositions of the present invention comprising from about 1% (w/w) to 30% (w/w) of solvent based on total weight of the composition.

In some embodiments, the topical compositions of the present invention comprise a) a low-dose clobetasol; b) an oil phase comprising at least one penetration enhancing agent, and a non-polymeric thickening agent; and c) an aqueous phase. In some embodiments, the topical compositions of the present invention comprise a) a low-dose clobetasol; b) an oil-in-water emulsion comprising a dispersed oil phase comprising at least one penetration enhancing agent, and a non-polymeric thickening agent, and a continuous aqueous phase; and c) one or more pharmaceutically acceptable excipients. In some embodiments, the topical compositions of the present invention comprise low-dose clobetasol.

In some embodiments, the topical compositions of the present invention are oil-in-water emulsions or water-in-oil emulsions. In some embodiments, the topical compositions comprise clobetasol are oil-in-water emulsions. In some embodiments, the topical compositions are oil-in-water emulsions comprising (a) a dispersed oil phase comprising at least one skin penetration enhancing agent, a non-polymeric thickening agent and a continuous aqueous phase; and (b) one or more pharmaceutically acceptable excipients.

In some embodiments, the topical composition of the present invention forms a depot on the skin forming an occlusive film, thereby extending the duration of active agent action while allowing “breathing” of the skin.

In some embodiment, the compositions of the present invention may have pH values ranging from about 3.0 to about 7.0 or from about 3.5 to about 6.0.

The compositions of the present invention can be dispensed in any dispensing device such as laminated tubes or lacquered aluminum tubes. Laminated tubes contains propylene glycol-free topical compositions, wherein the device is a lamitube comprised of 5 layers White PE, Ethylene acrylic acid (EAA), Aluminum foil, EAA, Virgin natural PE such that the composition is consistently discharge on application. Used. In an embodiment, the compositions of the present invention are dispensed in lacquered aluminum tubes which are very useful and very effective in storing the cream.

In some embodiments, the total patient population is a pediatric patient population. In some embodiments, the total patient population is composed of subjects that are male or female aged about 6 years to about 16 years and 11 months of age. In some embodiments, the total patient population is composed of subjects that are a male or female aged about 12 years to about 16 years and 11 months of age. In some embodiments, the total patient population is composed of subjects that are male or female under the age of 18 years. In some embodiments, the total patient population is composed of subjects that are a male or female under the age of 12 years.

In some embodiments, the pediatric subject is an individual. In some embodiments, the pediatric subject is a male or female aged about 6 years to about 16 years and 11 months of age. In some embodiments, the pediatric subject is a male or female aged about 12 years to about 16 years and 11 months of age. In some embodiments, the pediatric subject is a male or female under the age of 18 years. In some embodiments, the pediatric subject is a male or female under the age of 12 years.

In some embodiments, the term “subject” refers to a patient with psoriasis involving of at least about 5% body surface area or a patient with psoriasis involving of at least about 10% body surface area or a patient with psoriasis involving of more than about 10% body surface area. In some embodiments, the pediatric subject has an IGA score of less than, or equal to 3, a body surface area affected that is greater than or equal to about 10%, or a combination thereof.

In some embodiments, the present invention provides methods for the prophylaxis, amelioration or treatment of skin diseases or disorders such as psoriasis/psoriatic plaques, relief of the inflammatory and pruritic manifestations of steroid responsive dermatoses, erythema, contact sensitivity reactions, atopic dermatitis, seborrheic dermatitis, eczema, plaque psoriasis, erythrodermic psoriasis, psoriasis of the scalp, and other associated diseases or disorders in a pediatric subject in need thereof, by administering to said pediatric subject an effective amount of a topical composition comprising about 0.05% to about 0.045% (w/w) of clobetasol, an aqueous phase, an oil phase; about 5% of an emollient, about 0.05% of an antioxidant, about 10% of a solvent, about 6% of an emulsifier, and a preservative; wherein the aqueous phase is water and the water is at least 60% the total weight of the composition, wherein the oil phase comprises at least one penetration enhancing agent in an amount from about 0.01% to about 15.0% of the total weight of the composition and a non-polymeric thickening agent, wherein the subject has a decreased risk of hypothalamic-pituitary-adrenal (HPA) axis suppression, a decreased risk of side effects or a combination thereof.

In some embodiments of the present invention, psoriasis is mild to moderate plaque psoriasis. In some embodiments of the present invention, psoriasis is moderate to severe plaque psoriasis.

In some embodiments, the present invention includes topical compositions including a corticosteroid and their use in the treatment of psoriasis in a pediatric patient population. In some embodiments, the psoriasis is moderate to severe plaque psoriasis.

The compositions of the present invention can be applied directly onto affected areas of the skin, such as psoriatic plaques or dermatoses of a pediatric subject. Cream compositions, are applied in the form of film on the affected areas and, in embodiments, can provide release of the active agent for an extended duration of time.

In some embodiments, the methods of treating psoriasis in a pediatric subject involve topical administration of a composition comprising about 0.025% (w/w) of clobetasol to the subject, wherein said pediatric subject having psoriatic lesions involving at least about 5% body surface area or from about 5% to about 10% body surface area or more than about 10% body surface area. In some embodiments, the composition is comprises about 0.025% (w/w) clobetasol propionate.

In some embodiments, the topical compositions of the present invention provide a similar or improved therapeutic efficacy and/or reduced adverse effects of clobetasol in a pediatric subject. In some embodiments, the topical compositions of the present invention provide a similar or improved therapeutic efficacy and/or reduced adverse effect of clobetasol propionate in a pediatric subject. In some embodiments, at least 30% of the subjects treated with the topical composition of the present invention go from severe or very severe to moderate, or moderate to mild, minimal or almost clear or clear condition in the 15 days treatment.

In some embodiments, the method of treating psoriasis in a pediatric subject involves topical administration of a composition comprising about 0.05% to about 0.045% (w/w) of clobetasol, to the pediatric subject, once daily to the affected areas of the skin for a period of from about one day to two weeks, or twice daily for a period of from about one day to two weeks to the affected areas of the skin, or once daily to the affected areas of the skin for a period of from about one day to about four weeks, or twice daily to the affected areas of the skin for a period of from about one day to about four weeks. In some embodiments, the method of topical administration of the composition is twice daily for about four weeks or about 30 days. In some embodiments, the method of topical administration of the composition is twice daily for about two weeks or about 15 days. In some embodiments, the composition is comprises about 0.025% (w/w) clobetasol propionate.

In some embodiments, the methods of treating psoriasis in a pediatric patient involves topical administration of a composition comprising about 0.025% (w/w) clobetasol, to the pediatric subject, once daily to the affected areas of the skin for a period of from about one day to two weeks, or twice daily for a period of from about one day to two weeks to the affected areas of the skin, or once daily to the affected areas of the skin for a period of from about one day to about four weeks, or twice daily to the affected areas of the skin for a period of from about one day to about four weeks. In some embodiments, the method of topical administration of the composition is twice daily for about four weeks or about 30 days. In some embodiments, the method of topical administration of the composition is twice daily for about two weeks or about 15 days. In some embodiments, the composition is comprises about 0.025% (w/w) clobetasol propionate.

In some embodiments, the methods described herein comprise administering a topical composition comprising about 0.025% (w/w) clobetasol, once or twice daily with a dose of from about 1 gram to about 12 grams per day for a period of about one day to about two weeks. In another aspect, the composition comprises about 0.025% (w/w) of clobetasol propionate.

In some embodiments, the methods described herein comprise administering a topical composition comprising about 0.025% (w/w) clobetasol, once or twice daily with the dose of from about 0.1 mg to 3.15 mg per day of clobetasol for a period of about two weeks. In another aspect, clobetasol is clobetasol propionate.

In some embodiments, the methods described herein comprise administering a topical composition comprising about 0.025% (w/w) clobetasol, once or twice daily with the dose of from about 0.25 mg to 2.5 mg per day of clobetasol for a period of about two weeks. In another aspect, clobetasol is clobetasol propionate

In some embodiments, the topical compositions of the present invention can be administered in a total daily dose of more than about 2 grams/day, but not exceeding about 12 g/day for one week in the subject with eczema.

In some embodiments, the topical compositions of the present invention can be administered in a total weekly dose of more than about 60 g/week without causing clinically significant HPA axis suppression in the subject.

In some embodiments, the present invention relates to methods of treating psoriasis in a pediatric subject, said method comprises administering a topical composition comprising about 0.025% (w/w) of clobetasol, once or twice daily with a dose of from about 1 grams to about 7 grams per day of said topical composition for a period of about two weeks; wherein said method administers clobetasol from about 0.25 mg to about 2.5 mg per day; and wherein treating is substantially free of HPA axis suppression and said composition provides mean clobetasol plasma levels less than about 130 pg/mL. In another aspect, clobetasol is clobetasol propionate.

In some embodiments, the present invention relates to methods of treating psoriasis in a pediatric subject, said method comprises administering topical composition comprising about 0.025% (w/w) of clobetasol, once or twice daily with a dose of from about 1 grams to about 7 grams per day of said topical composition for a period of about two weeks; wherein said method administers clobetasol from about 0.25 mg to about 2.5 mg per day; and provides low percentage reduction of serum concentration of DHEAS and said composition provides mean clobetasol plasma levels less than about 130 pg/mL. In another aspect, clobetasol is clobetasol propionate.

In some embodiments, the present invention relates to methods of treating psoriasis in a pediatric subject, said method comprising administering a topical composition comprising about 0.025% (w/w) of clobetasol, once or twice daily with the a dose of from about 1 grams to about 7 grams per day of said topical composition for a period of two weeks; wherein said method administers clobetasol from about 1.25 mg to about 2.25 mg per day; and said method is substantially free of adverse effects. In another aspect, clobetasol is clobetasol propionate.

In some embodiments, post treatment plasma concentrations are measured post 8 weeks treatment period. In some embodiments, post treatment plasm concentrations are measured post 4 weeks treatment period. In some embodiments, post treatment plasm concentrations are measured post 15 days treatment period.

In some embodiments, the present invention provides methods of treating psoriasis in a pediatric subject, wherein the topical composition, comprising from about 0.025% (w/w) clobetasol propionate, is administered topically to the subject's affected skin area. In some embodiments, the post treatment mean clobetasol propionate plasma levels are less than about 150 pg/ml or the post treatment mean clobetasol propionate plasma levels are less than about 130 pg/ml, or the post treatment mean clobetasol propionate plasma levels are less than about 100 pg/ml or the post treatment mean clobetasol propionate plasma levels are less than about 75 pg/ml or the post treatment mean clobetasol propionate plasma levels are less than about 50 pg/ml or the post treatment mean clobetasol propionate plasma levels are less than about 25 pg/ml or the post treatment mean clobetasol propionate plasma levels are below quantifiable level.

In some embodiments, the topical compositions of the present invention provide mean clobetasol plasma levels in pediatric subjects, which are about 1 fold less than the levels of the topical composition comprising the highest approved topical dose of clobetasol i.e. 0.05% (w/w) or about 1.5 folds less than the levels of the topical composition comprising the highest approved topical dose of clobetasol i.e. 0.05% (w/w) or about 2.5 folds less than the levels of the topical composition comprising the highest approved topical dose of clobetasol i.e. 0.05% (w/w) or about 3.0 folds less than the levels of topical composition comprising the highest approved topical dose of clobetasol i.e. 0.05% (w/w) or about 3.5 folds less than the levels of topical composition comprising the highest approved topical dose of clobetasol i.e. 0.05% (w/w) clobetasol with a similar or improved therapeutic efficacy.

In some embodiments, the topical compositions of the present invention comprise a) a low-dose clobetasol; b) an oil phase comprising at least one penetration enhancing agent, and a non-polymeric thickening agent; and c) an aqueous phase; which provides mean clobetasol plasma levels insufficient to reduce serum levels of cortisol less than or equal to about 18 μg/dL in a pediatric subject.

In some embodiments, the topical compositions of the present invention comprise a) about 0.025% (w/w) clobetasol; b) an oil phase comprising at least one penetration enhancing agent, and a non-polymeric thickening agent; and c) an aqueous phase; which provides mean clobetasol plasma levels insufficient to reduce serum levels of cortisol less than or equal to about 18 μg/dL in a pediatric subject.

In some embodiments, the topical compositions of the present invention comprise a) about 0.025% (w/w) clobetasol propionate; b) an oil phase comprising at least one penetration enhancing agent, and a non-polymeric thickening agent; and c) an aqueous phase; which provides mean clobetasol propionate plasma levels insufficient to reduce serum levels of cortisol less than or equal to about 18 μg/dL in a pediatric subject.

In some embodiments, the topical compositions of present invention comprise from about 0.005% (w/w) to about 0.04% (w/w) of clobetasol, wherein said compositions provide and provides mean clobetasol plasma levels in the range of from about 130 pg/mL to 0 pg/mL in a pediatric subject, and the mean plasma concentrations are measured post eight weeks treatment or post four weeks treatment or post two weeks treatment in a pediatric subject.

In some embodiments, the topical compositions of the present invention comprise a) about 0.025% (w/w) clobetasol; b) an oil phase comprising at least one penetration enhancing agent, and a non-polymeric thickening agent; and c) an aqueous phase; and provide mean clobetasol plasma levels in the range of from about 130 pg/mL to 0 pg/mL in a pediatric subject.

In some embodiments, the topical compositions of the present invention comprise a) about 0.025% (w/w) clobetasol; b) an oil-in-water emulsion comprising a dispersed oil phase comprising at least one penetration enhancing agent, and a non-polymeric thickening agent, and a continuous aqueous phase; and c) one or more pharmaceutically acceptable excipients; and provides mean clobetasol plasma levels in the range of from about 130 pg/mL to 0 pg/mL in a pediatric subject.

In some embodiments, the topical compositions of the present invention comprise a) about 0.025% (w/w) clobetasol propionate; b) an oil phase comprising at least one penetration enhancing agent, and a non-polymeric thickening agent; and c) an aqueous phase; and provides mean clobetasol propionate plasma levels in the range of from about 130 pg/mL to 0 pg/mL in a pediatric subject.

In some embodiments, the topical compositions of the present invention comprise a) about 0.025% (w/w) clobetasol propionate; b) an oil-in-water emulsion comprising a dispersed oil phase comprising at least one penetration enhancing agent, and a non-polymeric thickening agent, and a continuous aqueous phase; and c) one or more pharmaceutically acceptable excipients; and provide mean clobetasol propionate plasma levels in the range of from about 130 pg/mL to 0 pg/mL in a pediatric subject. In some embodiments, clobetasol propionate plasma levels are in the range of from about 120 pg/mL to about 20 pg/mL or in the range of 100 pg/mL to 20 pg/mL.

In some embodiments, the topical compositions of the present invention comprise a) about 0.025% (w/w) clobetasol propionate; b) an oil-in-water emulsion comprising a dispersed oil phase comprising at least one penetration enhancing agent, and a non-polymeric thickening agent, and a continuous aqueous phase; and c) one or more pharmaceutically acceptable excipients; which provides post treatment mean clobetasol propionate plasma levels in the range of from about 150 pg/mL to 0 pg/mL in a pediatric subject. In some embodiments, clobetasol propionate plasma levels are in the range of about 130 pg/mL to 0 pg/mL or about 100 pg/mL to about 10 pg/mL.

Topical corticosteroid use typically results in adverse effects on the human endocrine system. High potency corticosteroids have a high incidence of systemic side effects such as reversible suppression of hypothalamus-pituitary-adrenal (HPA) axis. Topical corticosteroids that are absorbed systemically typically result in HPA axis suppression. The HPA axis suppression represents a critical safety issue in topical corticosteroid therapy. HPA axis suppression is generally evaluated by certain parameters such as levels of cortisol in a subject's blood during the treatment schedule. The cortisol levels are determined by the ACTH (cosyntropin) stimulation test. The ACTH stimulation test measures how the adrenal glands respond to adrenocorticotropic hormone (ACTH). ACTH is a hormone produced in the pituitary gland that stimulates the adrenal glands to release a hormone called cortisol. The man-made form of ACTH is called cosyntropin. The normal level of cortisol is less than 18 mcg/dL in a normal subject and cortisol level goes higher than 18 to 20 micrograms per deciliter (mcg/dL) after ACTH injection to the subject.

Clobetasol propionate is a highly potent topical corticosteroid, which is known to have effects on the endocrine system which suppresses the HPA axis at doses as low as 2 grams per day. Shortcomings of currently approved therapy include the necessity for periodic evaluation for HPA axis suppression and modification in dosing and administrating schedules when HPA axis suppression occurs.

The topical compositions of the present invention and their use in the methods described herein do not result in significant adverse effect on endocrine system, when applied twice daily for 15 days (2 weeks) in the pediatric subjects having an affected body surface area of at least 10% excluding face, scalp, groin, axillae and other intertriginous areas.

The term “substantially free of adverse effects” as used herein indicates that at least about 90% of total patient population does not have adverse effects resultant from clobetasol based compositions or about 80% of total patient population does not have adverse effects or about 75% of total patient population does not have adverse effects or at least about 70% of total patient population does not have adverse effects or at least about 60% of total patient population does not have adverse effects. In some embodiments, the compositions of the present application provides therapeutic efficacy and do not exhibit significant adverse effects on the endocrine system as described herein and as known to those of ordinary skill in the art. In an alternative, the term “substantially free of adverse effects” as used herein indicates that administration of the composition to an individual pediatric subject results in an at least about 60% chance that the patient will not develop adverse effects. Further, it has been observed that the topical pharmaceutical compositions of the present invention, which are free of propylene glycol, are non-irritating, non-toxic, and well tolerated and are free of any undesired attributes, thereby providing a high degree of patient compliance in pediatric subjects.

In some embodiments, the an adverse effects of clobetasol include but are not limited to adverse effect on the endocrine system. In some embodiments, the adverse effect on the endocrine system is HPA axis suppression. In some embodiments, the adverse effects of clobetasol include but are not limited to Cushing's syndrome, hyperglycemia, and glycosuria, linear growth retardation, delayed weight gain, and intracranial hypertension. In some embodiments, local adverse reactions include but are not limited to striae and skin atrophy. In some embodiments, manifestations of adrenal suppression in children include low plasma cortisol levels and absence of response to ACTH stimulation. Manifestations of intracranial hypertension include bulging fontanelles, headaches, and bilateral papilledema.

In some embodiments, the topical compositions of the present invention comprising 0.025% (w/w) clobetasol provide the therapeutically beneficial effects of clobetasol while being substantially free of an adverse effect of clobetasol wherein said adverse effect is HPA axis suppression in a pediatric subject.

In yet other embodiments, the term “substantially free” as used herein indicates that the specified effect (for example HPA axis suppression) occurs in not more than about 15% of subjects treated with the compositions described herein, or not more than about 10% of subjects treated with the compositions described herein, or not more than about 5% of subjects treated with the compositions described herein, or not more than more than about 1% of subjects treated with the compositions described herein, or not more than about 0% of subjects treated with the compositions described herein, or completely free of the specified effect. In yet other embodiments, the term “substantially free” as used herein indicates that plasma concentrations of clobetasol that are insufficient to reduce serum cortisol levels less than or equal to 18 μg/dL.

In some embodiments, treating the pediatric patient subject results in a risk of HPA axis suppression that is similar to the risk of HPA axis suppression in an adult patient. In some embodiments, treating a pediatric patient population results in a risk of HPA axis suppression that is similar to the risk of HPA axis suppression in an adult patient population.

In some embodiments, treating moderate to severe plaque psoriasis in the pediatric subject results in at least 70% of subjects not having hypothalamic-pituitary-adrenal (HPA) axis suppression. In some embodiments, treating moderate to severe plaque psoriasis in the pediatric subject results in at least 80% of subjects not having hypothalamic-pituitary-adrenal (HPA) axis suppression. In some embodiments, treating moderate to severe plaque psoriasis in the pediatric subject results in at least 85% of subjects not having hypothalamic-pituitary-adrenal (HPA) axis suppression. In some embodiments, treating moderate to severe plaque psoriasis in the pediatric subject results in the subject being substantially free of adverse effects. In some embodiments, the adverse effect is skin irritation, vein collapse, itching, burning, stinging or a combination thereof. In some embodiments, treating moderate to severe plaque psoriasis in a pediatric subject results in substantially no hypothalamic-pituitary-adrenal (HPA) axis suppression. In some embodiments, the adverse effect is selected from Cushing's syndrome, hyperglycemia, and glycosuria, linear growth retardation, delayed weight gain, and intracranial hypertension and a combination thereof.

For example, about 90% of total patient population does not have HPA axis suppression or about 80% of total patient population does not have HPA axis suppression or about 75% of total patient population does not have HPA axis suppression or at least about 70% of total patient population does not have HPA axis suppression or at least about 60% of total patient population does not have HPA axis suppression. In some embodiments, “substantially free of HPA axis suppression” as used herein means at least about 90% of total patient population does not have HPA axis suppression or about 80% of total patient population does not HPA axis suppression or about 75% of total patient population does not HPA axis suppression or at least about 70% of total patient population does not have HPA axis suppression or at least about 60% of total patient population does not have HPA axis suppression.

In some embodiments, treating moderate to severe plaque psoriasis in a pediatric subject results in an at least about 80% chance that the subject will not develop hypothalamic-pituitary-adrenal (HPA) axis suppression. In some embodiments, treating moderate to severe plaque psoriasis in a pediatric subject results in an at least about 80% chance that the subject will not develop hypothalamic-pituitary-adrenal (HPA) axis suppression. In some embodiments, treating moderate to severe plaque psoriasis in a pediatric subject results in an at least about 85% chance that the subject will not develop hypothalamic-pituitary-adrenal (HPA) axis suppression. In some embodiments, treating moderate to severe plaque psoriasis in a pediatric subject results in at least about 90% chance that the subject will not develop hypothalamic-pituitary-adrenal (HPA) axis suppression. In some embodiments, treating moderate to severe plaque psoriasis in the pediatric subject results in the subject having the same risk for adverse effects as an adult subject. In some embodiments, treating moderate to severe plaque psoriasis in the pediatric subject results in the subject being substantially free of adverse effects. In some embodiments, the adverse effect is skin irritation, vein collapse, itching, burning, stinging or a combination thereof. In some embodiments, treating moderate to severe plaque psoriasis in a pediatric subject results in substantially no hypothalamic-pituitary-adrenal (HPA) axis suppression. In some embodiments, the adverse effect is selected from Cushing's syndrome, hyperglycemia, and glycosuria, linear growth retardation, delayed weight gain, and intracranial hypertension and a combination thereof.

For example, the pediatric subject has about 90% chance to not develop HPA axis suppression, about 85% chance to not develop HPA axis suppression, or about 80% chance to not develop HPA axis suppression or about 75% chance to not develop HPA axis suppression or about 70% chance to not develop HPA axis suppression or about 60% chance to not develop HPA axis suppression. In some embodiments, “substantially free of HPA axis suppression” as used herein means at an individual pediatric subject has a 90% chance to not have HPA axis suppression or about 80% chance to not have HPA axis suppression or about 75% chance to not have HPA axis suppression or about 70% chance to not have HPA axis suppression or about 60% chance to not have HPA axis suppression.

In some embodiments, the topical compositions of the present invention comprise a) a low-dose clobetasol; b) an oil phase comprising at least one penetration enhancing agent, and a non-polymeric thickening agent; and c) an aqueous phase; which is substantially free of adverse effects, wherein the adverse effects include an effect on the endocrine system such as adrenal gland which in turn causes HPA axis suppression in a pediatric subject. In some embodiments, administration of the topical compositions results in HPA axis suppression without any clinical symptoms.

In some embodiments, the topical compositions of the present invention comprise a) a low-dose clobetasol; b) an oil phase comprising at least one penetration enhancing agent, and a non-polymeric thickening agent; and c) an aqueous phase; which is substantially free of adverse effects, wherein the adverse effects include reduced levels of dehydroepiandrosterone sulfate (DHEAS) in a pediatric subject.

In some embodiments, the topical compositions of the present invention comprise a) about 0.025% (w/w) clobetasol; b) an oil phase comprising at least one penetration enhancing agent, and a non-polymeric thickening agent; and c) an aqueous phase; and is substantially free of adverse effects, wherein the adverse effects include an effect on endocrine system such as adrenal gland stimulation which in turn causes HPA axis suppression in a pediatric subject. In some embodiments, administration of the topical compositions results in HPA axis suppression without any clinical symptoms.

In some embodiments, the topical compositions of the present invention comprise a) about 0.025% (w/w) clobetasol; b) an oil phase comprising at least one penetration enhancing agent, and a non-polymeric thickening agent; and c) an aqueous phase; which is substantially free of adverse effects, wherein the adverse effects include reduced levels of dehydroepiandrosterone sulfate (DHEAS) in a pediatric subject.

In some embodiments, the topical compositions of the present invention comprise a) about 0.025% (w/w) clobetasol propionate; b) an oil phase comprising at least one penetration enhancing agent, and a non-polymeric thickening agent; and c) an aqueous phase; which is substantially free of adverse effects, wherein the adverse effects include an effect on endocrine system such as adrenal gland stimulation which in turn causes HPA axis suppression in a pediatric subject. In some embodiments, administration of the topical compositions results in HPA axis suppression without any clinical symptoms.

In some embodiments, the topical compositions of the present invention comprise a) about 0.025% (w/w) clobetasol propionate; b) an oil phase comprising at least one penetration enhancing agent, and a non-polymeric thickening agent; and c) an aqueous phase; which is substantially free of adverse effects, wherein said adverse effects include reduced levels of dehydroepiandrosterone sulfate (DHEAS) in a pediatric subject.

In some embodiments, the topical compositions of the present invention comprise a) about 0.025% (w/w) clobetasol propionate; b) an oil phase comprising at least one penetration enhancing agent, and a non-polymeric thickening agent; and c) an aqueous phase; which is substantially free of adverse effects, wherein the adverse effects are HPA axis suppression and reduced levels of dehydroepiandrosterone sulfate (DHEAS) in a pediatric subject. In some embodiments, administration of the topical compositions results in HPA axis suppression without any clinical symptoms.

In some embodiments, the topical compositions of the present invention comprise a) about 0.025% (w/w) clobetasol propionate; b) an oil phase comprises at least one penetration enhancing agent, and a non-polymeric thickening agent; and c) an aqueous phase; which is substantially free of adverse effects, wherein the adverse effects are HPA axis suppression and reduced levels of dehydroepiandrosterone sulfate (DHEAS) in a pediatric subject. In some embodiments, administration of the topical compositions results in HPA axis suppression without any clinical symptoms.

In some embodiments, the compositions of the present invention provide mean plasma concentration which is insufficient to cause clinically significant HPA axis suppression in a pediatric subject. In some embodiments, administration of the topical compositions results in HPA axis suppression without any clinical signs or symptoms. The clinical signs and symptoms of HPA axis suppression include but are not limited to the inability to mount a stress response in face of infection or other stresses, constitutional symptoms of hypotension, tachycardia. In some embodiments, the signs and symptoms of HPA axis suppression are due to low reserve function. In some embodiments, the signs and symptoms of HPA axis suppression are due to exogenously administered steroids can cause signs and symptoms of excess cortisol like growth retardation, bone problems (e.g. aseptic necrosis) glaucoma, cataracts, and skin changes.

In some embodiments, the topical compositions of the present invention comprise low-dose clobetasol, and when administered to a pediatric subject is substantially free of HPA axis suppression.

In some embodiments, the topical compositions of the present invention comprise about 0.025% (w/w) clobetasol, and when administered to a pediatric subject is substantially free of HPA axis suppression.

In some embodiments, the topical compositions of the present invention comprise about 0.025% (w/w) clobetasol propionate, and when administered to a pediatric subject is substantially free of HPA axis suppression in the subject.

In some embodiments, the percentage reduction in DHEA or DHEAS serum levels is a parameter for adverse effects of steroid drugs such as clobetasol. The topical compositions of the present invention provides lower percentage reduction in serum concentration of DHEA or DHEAS. In some embodiments, the topical compositions of the present invention comprise a low-dose clobetasol, wherein said composition provides low percentage reduction of the serum concentration of DHEAS.

In some embodiments, the topical compositions of the present invention comprise 0.025% (w/w) clobetasol, wherein said compositions result in a low percentage reduction of serum concentration of DHEAS, In some embodiments, the percentage reduction is less than about 15%.

In some embodiments, the topical compositions of the present invention comprise 0.025% (w/w) clobetasol propionate, wherein said composition results in a low percentage reduction of serum concentration of DHEAS. In some embodiments, the percentage reduction is less than about 15%.

In some embodiments, the topical compositions of the present invention comprise about 0.025% (w/w) clobetasol, wherein said composition results in a low percentage of reduction of serum concentration of DHEAS. In some embodiments, the percentage reduction is less than about 15%.

In some embodiments, the topical compositions of the present invention comprise about 0.025% (w/w) clobetasol propionate, wherein said composition provides post treatment mean plasma concentration of clobetasol propionate less than about 150 pg/ml, and results in a low percentage of reduction of serum concentration of DHEAS in a pediatric subject. In some embodiments, the percentage reduction is less than about 15%.

In some embodiments, the topical compositions of the present invention comprise about 0.025% (w/w) clobetasol, wherein said composition results in a post treatment mean plasma concentration of clobetasol less than about 150 pg/ml, and results in a low percentage of reduction of serum concentration of DHEAS and is substantially free of HPA axis suppression in a pediatric subject.

In some embodiments, the topical compositions of the present invention comprise about 0.025% (w/w) clobetasol propionate, wherein said composition provides a post treatment mean plasma concentration of clobetasol propionate less than about 150 pg/ml, and provides low percentage of reduction of serum concentration of DHEAS and is substantially free of HPA axis suppression in a pediatric subject.

In some embodiments, the present invention relates to methods of treating psoriasis in a pediatric subject, said method comprising the topical administration of a composition, comprising about 0.025% (w/w) of clobetasol, to the subject's affected areas of the skin; wherein said composition is substantially free of adverse effects.

In some embodiments, the methods of treating psoriasis in a pediatric subject, comprise the topical administration of a composition comprising about 0.025% (w/w) of clobetasol propionate, to the pediatric subject once or twice daily to affected areas of the skin for about two weeks to about four weeks; wherein said composition is substantially free of adverse effects and said composition provides mean clobetasol propionate plasma levels less than about 130 pg/mL.

In some embodiments, the method of treating psoriasis in a pediatric subject, comprises the topical administration of a composition comprising about 0.025% (w/w) of clobetasol propionate, to the subject once or twice daily to affected areas of the skin for about two weeks to about four weeks; wherein said composition is substantially free of adverse effects and said composition provides post treatment mean clobetasol propionate plasma levels less than about 150 pg/mL.

In some embodiments, the method of treating psoriasis in a pediatric subject, comprises a the topical administration of a composition comprising about 0.025% (w/w) of clobetasol propionate, to the subject once or twice daily to affected areas of the skin for about two weeks to about four weeks; wherein said composition is substantially free of adverse effects and said composition provides post treatment mean clobetasol propionate plasma levels less than about 150 pg/mL.

In some embodiments, the method of treating psoriasis in a pediatric subject, comprises the topical administration of a composition comprising about 0.025% (w/w) of clobetasol propionate, to the subject once or twice daily to affected areas of the skin for about two week to about four weeks; wherein said composition is substantially free of adverse effects and said composition provides post treatment mean clobetasol propionate plasma levels about from 130 pg/mL to 0 pg/ml.

In some embodiments, the methods of treating psoriasis in a pediatric subject, comprise the topical administration of a composition comprising about 0.025% (w/w) of clobetasol propionate, to the subject once or twice daily to affected areas of the skin for a period of from about two weeks to about four weeks; wherein said composition is substantially free of adverse effects and said composition provides post treatment mean clobetasol propionate plasma levels about from 150 pg/mL to 0 pg/ml.

In some embodiments, the methods of treating psoriasis in a pediatric subject, comprise the topical administration of a composition comprising about 0.025% (w/w) of clobetasol propionate, to the subject once or twice daily to affected areas of the skin for a period of about two weeks; wherein said composition is substantially free of adverse effects and said composition provides post treatment mean clobetasol propionate plasma levels about from 150 pg/mL to 0 pg/ml.

In some embodiments, the present invention relates to methods of treating psoriasis in a pediatric subject having psoriatic lesions more than about 10% body surface area, said method comprising topical administration of a composition, comprising about 0.025% (w/w) of clobetasol propionate, to the subject's affected areas of the skin twice daily for a treatment period of about two weeks; wherein said method provides substantially free of adverse effects. In some embodiments, the topical administration of a composition comprising about 0.025% (w/w) clobetasol propionate, involves once or twice daily for a treatment period of two weeks. In some embodiments, the adverse effects include HPA axis suppression and/or reduction in DHEAS.

In some embodiments, the present invention provides methods of treating psoriasis in a pediatric subject, wherein the topical composition, comprising a low-dose clobetasol administered topically to the subject's affected skin area and is substantially free of adverse effects. In some embodiments, the composition comprises about 0.025% (w/w) clobetasol. In some embodiments, the composition comprises about 0.025% (w/w) clobetasol propionate.

In some embodiments, the present invention provides methods of treating psoriasis in a pediatric subject, wherein said topical composition, comprising about 0.025% of clobetasol, is administered topically to the subject's affected skin area and said treatment provides low percentage of reduction in serum concentration of dehydroepiandrosterone sulfate (DHEAS). In some embodiments, the composition is comprises about 0.025% (w/w) clobetasol propionate.

In some embodiments, the present invention provides methods of treating psoriasis in a pediatric subject, wherein said topical composition, comprising about 0.025% (w/w) of clobetasol, is administered topically to the subject's affected skin area and said treatment provides lower percentage of reduction in serum concentration of dehydroepiandrosterone sulfate (DHEAS) and is substantially free of HPA axis suppression. In some embodiments, the composition comprises about 0.025% (w/w) clobetasol propionate.

In some embodiments, the present invention provides methods of treating a pediatric subject having psoriatic lesions equal to or more than about 10% of the body surface area, wherein the topical composition comprising low-dose clobetasol is administered to said subject's surface area and such treatment provides substantially no hypothalamic-pituitary-adrenal (HPA) axis suppression, low percentage of reduction in serum concentration of dehydroepiandrosterone sulfate (DHEAS). In some embodiments, the composition comprises about 0.025% (w/w) clobetasol. In some embodiments, the composition comprises about 0.025% (w/w) clobetasol propionate.

In some embodiments, the present invention provides methods of treating psoriasis in a pediatric subject, wherein the topical composition, comprising low-dose clobetasol is administered topically to the subject's affected skin area and said treatment provides a lower percentage of reduction in serum concentration of dehydroepiandrosterone sulfate (DHEAS) and low post treatment clobetasol mean plasma concentration in the subjects with no HPA axis suppression as compared to that of subjects with significant HPA axis suppression.

In some embodiments, the present invention provides methods of treating psoriasis in a pediatric subject, wherein the topical composition, comprising about 0.025% (w/w) clobetasol propionate, is administered topically to the subject's affected skin area and said treatment provides a lower percentage of reduction in serum concentration of dehydroepiandrosterone sulfate (DHEAS) and low post treatment clobetasol propionate mean plasma concentration in the subjects with no HPA axis suppression as compared to that of subjects with significant HPA axis suppression.

In some embodiments, the present invention provides methods of treating psoriasis in a pediatric subject, wherein the topical composition, comprising low-dose clobetasol, is administered topically to the subject's affected skin area and said treatment provides a lower percentage of reduction in serum concentration of dehydroepiandrosterone sulfate (DHEAS) and low post treatment clobetasol mean plasma concentration in the subjects with significantly no HPA axis suppression as compared to that of subjects with significant HPA axis suppression.

In some embodiments, the present invention provides methods of treating psoriasis in a pediatric subject, wherein the topical composition, comprising about 0.025% (w/w) clobetasol, is administered topically to the subject's affected skin area once or twice daily for a period of at least one day to about four weeks; and said treatment provides lower percentage of reduction in serum concentration of dehydroepiandrosterone sulfate (DHEAS) and low post treatment clobetasol plasma concentration in the subjects with significantly no HPA axis suppression as compared to that of subjects with significant HPA axis suppression.

In some embodiments, the present invention provides methods of treating psoriasis in a pediatric subject, wherein the topical composition, comprising about 0.025% (w/w) clobetasol propionate, is administered topically to the subject's affected skin area for a period of at least one day to about four weeks; and said treatment provides lower percentage of reduction in serum concentration of dehydroepiandrosterone sulfate (DHEAS) and low post treatment clobetasol propionate plasma concentration in the subjects with significantly no HPA axis suppression as compared to that of subjects with significant HPA axis suppression.

In some embodiments, the present invention provides a method of treating psoriasis in a pediatric subject, wherein the topical composition, comprising about 0.025% (w/w) clobetasol, is administered topically to the subject's affected skin area for a period of at least one day to about two weeks; and said treatment provides lower percentage of reduction in serum concentration of dehydroepiandrosterone sulfate (DHEAS) and low post treatment clobetasol plasma concentration in the subjects with no HPA axis suppression as compared to that of subjects with significant HPA axis suppression.

In some embodiments, the present invention provides methods of treating psoriasis in a pediatric subject, wherein the topical composition, comprising about 0.025% (w/w) clobetasol propionate, is administered topically to the subject's affected skin area for a period of at least one day to about two weeks; and said treatment provides lower percentage of reduction in serum concentration of dehydroepiandrosterone sulfate (DHEAS) and low post treatment clobetasol propionate plasma concentration in the subjects with no HPA axis suppression as compared to that of subjects with significant HPA axis suppression.

In some embodiments, of the present invention, post treatment mean clobetasol propionate plasma levels are significantly less in the subjects having no HPA axis suppression as compared to that of subjects with HPA axis suppression.

In some embodiments, the compositions of the present invention provide significantly greater percent reduction in serum DHEAS concentration and a significantly greater mean post-treatment clobetasol propionate plasma concentration in subjects HPA axis suppression than subjects without HPA axis suppression.

In another embodiment, treatment with the topical compositions disclosed herein results in a percentage reduction in serum concentration of dehydroepiandrosterone sulfate (DHEAS) in pediatric subjects with HPA axis suppression that is significantly greater than in pediatric subjects without HPA axis suppression.

In some embodiments, the methods of treating psoriasis comprise administering the topical composition of the present invention comprising about 0.025% (w/w) clobetasol and wherein more than about 2 grams/day of clobetasol can be administered without HPA axis suppression. In some embodiments, the clobetasol is clobetasol propionate.

In some embodiments, the methods of treating psoriasis comprise administering the topical composition of the present invention comprising about 0.025% (w/w) clobetasol and wherein more than about 2 grams/day of clobetasol can be administered with a lower percentage reduction of serum concentration of DHEAS. In some embodiments, the clobetasol is clobetasol propionate.

In some embodiments, the compositions of the present invention result in a mean plasma concentration which provides lower percentage reduction in serum levels of DHEAS. In some embodiments, the compositions of the present invention result in mean plasma concentrations which are substantially free of HPA axis suppression and provides lower percentage reduction in serum levels of DHEAS in a pediatric subject.

In some embodiments, the topical compositions of the present invention comprise a low-dose clobetasol which provides post treatment mean clobetasol plasma concentration, which is substantially free of HPA axis suppression; and/or lower percentage reduction in serum levels of DHEAS in a pediatric subject.

In some embodiments, the topical compositions of the present invention comprise about 0.025% (w/w) clobetasol and result in post treatment mean clobetasol plasma concentrations, which are substantially free of HPA axis suppression; and/or lower percentage reduction in serum levels of DHEAS in a pediatric subject.

In some embodiments, the topical compositions of the present invention comprise about 0.025% (w/w) clobetasol propionate and result in post treatment mean clobetasol plasma concentrations, which are substantially free of HPA axis suppression; and/or lower percentage reduction in serum levels of DHEAS in a pediatric subject.

In some embodiments, the topical compositions of the present invention comprise about 0.025% (w/w) clobetasol propionate and result in post treatment mean clobetasol propionate plasma concentrations, which are substantially free of HPA axis suppression; or lower percentage reduction in serum levels of DHEAS in a pediatric subject.

In some embodiments, the topical compositions of present invention comprise about 0.025% (w/w) clobetasol propionate and result in mean clobetasol propionate plasma levels less than or equal to about 130 pg/ml or 129 or 128 or 127 or 126 or 125 or 124 or 123 or 122 or 121 or 120 or 119 or 118 or 117 or 116 or 115 or 114 or 113 or 112 or 111 or 110 or 109 or 108 or 107 or 106 or 105 or 104 or 103 or 102 or 101 or 100 or 99 or 98 or 97 or 96 or 95 or 94 or 93 or 92 or 91 or 90 or 89 or 88 or 87 or 86 or 85 or 84 or 83 or 82 or 81 or 80 or 79 or 78 or 77 or 76 or 75 or 74 or 73 or 72 or 71 or 70 or 69 or 68 or 67 or 66 or 65 or 64 or 63 or 62 or 61 or 60 or 59 or 58 or 57 or 56 or 55 or 54 or 53 or 52 or 51 or 50 or 49 or 48 or 47 or 46 or 45 or 44 or 43 or 42 or 41 or 40 or 39 or 38 or 37 or 36 or 35 or 34 or 33 or 32 or 31 or 30 or 29 or 28 or 27 or 26 or 25 or 24 or 23 or 22 or 21 or 20 or 19 or 18 or 17 or 16 or 15 or 14 or 13 or 12 or 11 or 10 or 9 or 8 or 7 or 6 or 5 or 4 or 3 or 2 or 1 or 0 pg/mL in a pediatric subject when administered for 15 days and are substantially free of HPA axis suppression.

In some embodiments, the topical compositions of present invention comprise about 0.025% (w/w) clobetasol propionate and result in mean clobetasol propionate plasma levels less than or equal to about 130 pg/ml or 129 or 128 or 127 or 126 or 125 or 124 or 123 or 122 or 121 or 120 or 119 or 118 or 117 or 116 or 115 or 114 or 113 or 112 or 111 or 110 or 109 or 108 or 107 or 106 or 105 or 104 or 103 or 102 or 101 or 100 or 99 or 98 or 97 or 96 or 95 or 94 or 93 or 92 or 91 or 90 or 89 or 88 or 87 or 86 or 85 or 84 or 83 or 82 or 81 or 80 or 79 or 78 or 77 or 76 or 75 or 74 or 73 or 72 or 71 or 70 or 69 or 68 or 67 or 66 or 65 or 64 or 63 or 62 or 61 or 60 or 59 or 58 or 57 or 56 or 55 or 54 or 53 or 52 or 51 or 50 or 49 or 48 or 47 or 46 or 45 or 44 or 43 or 42 or 41 or 40 or 39 or 38 or 37 or 36 or 35 or 34 or 33 or 32 or 31 or 30 or 29 or 28 or 27 or 26 or 25 or 24 or 23 or 22 or 21 or 20 or 19 or 18 or 17 or 16 or 15 or 14 or 13 or 12 or 11 or 10 or 9 or 8 or 7 or 6 or 5 or 4 or 3 or 2 or 1 or 0 pg/mL in a pediatric subject when administered for 15 days and provides lower percentage reduction in DHEAS serum concentration.

In some embodiments, the topical compositions of present invention comprise about 0.025% (w/w) clobetasol propionate and result in post treatment mean clobetasol propionate plasma concentration less than or equal to about 150 pg/ml or 149 or 148 or 147 or 146 or 145 or 144 or 143 or 142 or 141 or 140 or 139 or 138 or 137 or 136 or 135 or 134 or 133 or 132 or 131 or 131 or 129 or 128 or 127 or 126 or 125 or 124 or 123 or 122 or 121 or 120 or 119 or 118 or 117 or 116 or 115 or 114 or 113 or 112 or 111 or 110 or 109 or 108 or 107 or 106 or 105 or 104 or 103 or 102 or 101 or 100 or 99 or 98 or 97 or 96 or 95 or 94 or 93 or 92 or 91 or 90 or 89 or 88 or 87 or 86 or 85 or 84 or 83 or 82 or 81 or 80 or 79 or 78 or 77 or 76 or 75 or 74 or 73 or 72 or 71 or 70 or 69 or 68 or 67 or 66 or 65 or 64 or 63 or 62 or 61 or 60 or 59 or 58 or 57 or 56 or 55 or 54 or 53 or 52 or 51 or 50 or 49 or 48 or 47 or 46 or 45 or 44 or 43 or 42 or 41 or 40 or 39 or 38 or 37 or 36 or 35 or 34 or 33 or 32 or 31 or 30 or 29 or 28 or 27 or 26 or 25 or 24 or 23 or 22 or 21 or 20 or 19 or 18 or 17 or 16 or 15 or 14 or 13 or 12 or 11 or 10 or 9 or 8 or 7 or 6 or 5 or 4 or 3 or 2 or 1 or 0 pg/mL in a pediatric subject when administered for 15 days, and are substantially free of HPA axis suppression.

In some embodiments, the topical compositions of present invention comprise about 0.025% (w/w) clobetasol propionate and result in post treatment mean clobetasol propionate plasma concentration less than or equal to about 150 pg/ml or 149 or 148 or 147 or 146 or 145 or 144 or 143 or 142 or 141 or 140 or 139 or 138 or 137 or 136 or 135 or 134 or 133 or 132 or 131 or 131 or 129 or 128 or 127 or 126 or 125 or 124 or 123 or 122 or 121 or 120 or 119 or 118 or 117 or 116 or 115 or 114 or 113 or 112 or 111 or 110 or 109 or 108 or 107 or 106 or 105 or 104 or 103 or 102 or 101 or 100 or 99 or 98 or 97 or 96 or 95 or 94 or 93 or 92 or 91 or 90 or 89 or 88 or 87 or 86 or 85 or 84 or 83 or 82 or 81 or 80 or 79 or 78 or 77 or 76 or 75 or 74 or 73 or 72 or 71 or 70 or 69 or 68 or 67 or 66 or 65 or 64 or 63 or 62 or 61 or 60 or 59 or 58 or 57 or 56 or 55 or 54 or 53 or 52 or 51 or 50 or 49 or 48 or 47 or 46 or 45 or 44 or 43 or 42 or 41 or 40 or 39 or 38 or 37 or 36 or 35 or 34 or 33 or 32 or 31 or 30 or 29 or 28 or 27 or 26 or 25 or 24 or 23 or 22 or 21 or 20 or 19 or 18 or 17 or 16 or 15 or 14 or 13 or 12 or 11 or 10 or 9 or 8 or 7 or 6 or 5 or 4 or 3 or 2 or 1 or 0 pg/mL in a pediatric subject when administered for 15 days, and provides lower percentage reduction in DHEAS serum concentration.

In some embodiments, the topical compositions of the present invention comprise a) about 0.025% (w/w) clobetasol propionate; b) an oil phase comprising at least one penetration enhancing agent, and a non-polymeric thickening agent; and c) an aqueous phase; and provides median clobetasol propionate plasma levels insufficient to reduce serum levels of cortisol less than or equal to about 18 μg/dL in a pediatric subject.

In some embodiments, the present invention relates to a methods for prophylaxis, amelioration, or treatment of psoriasis, relief of the inflammatory and pruritic manifestations of steroid responsive dermatoses, erythema, contact sensitivity reactions, and other associated diseases or disorders, by administering to a pediatric subject, a low-dose of clobetasol, to the affected area of the skin once or twice daily at least for one day; wherein said composition is substantially free of HPA axis suppression in said pediatric subject. In some embodiments, low-dose clobetasol is about 0.025% (w/w) clobetasol. In some embodiments, low-dose clobetasol is about 0.025% (w/w) clobetasol propionate.

In some embodiments, the present invention relates to methods for the prophylaxis, amelioration, or treatment of psoriasis, relief of the inflammatory and pruritic manifestations of steroid responsive dermatoses, erythema, contact sensitivity reactions, and other associated diseases or disorders, by administering to a pediatric subject, a low-dose of clobetasol, to the affected area of the skin once or twice daily up to about two weeks; wherein said composition is substantially free of HPA axis suppression. In some embodiments, low-dose clobetasol is about 0.025% (w/w) clobetasol. In some embodiments, low-dose clobetasol is about 0.025% (w/w) clobetasol propionate.

In some embodiments, the present invention relates to methods for the prophylaxis, amelioration, or treatment of psoriasis, relief of the inflammatory and pruritic manifestations of steroid responsive dermatoses, erythema, contact sensitivity reactions, and other associated diseases or disorders, by administering to a pediatric subject, a low-dose of clobetasol, to the affected area of the skin once or twice daily up to about four weeks; wherein said composition is substantially free of HPA axis suppression. In some embodiments, low-dose clobetasol is about 0.025% (w/w) clobetasol. In some embodiments, low-dose clobetasol is about 0.025% (w/w) clobetasol propionate.

In some embodiments, the present invention relates to methods for the prophylaxis, amelioration, or treatment of psoriasis, relief of the inflammatory and pruritic manifestations of steroid responsive dermatoses, erythema, contact sensitivity reactions, and other associated diseases or disorders, by administering to a pediatric subject, a low-dose clobetasol, to the affected area of the skin once or twice daily at least for one day; wherein said composition provides low percentage of reduction in DHEAS. In some embodiments, low-dose clobetasol is about 0.025% (w/w) clobetasol. In some embodiments, low-dose clobetasol is about 0.025% (w/w) clobetasol propionate.

In some embodiments, the present invention relates to methods for the prophylaxis, amelioration, or treatment of psoriasis, relief of the inflammatory and pruritic manifestations of steroid responsive dermatoses, erythema, contact sensitivity reactions, and other associated diseases or disorders, by administering to a pediatric subject, a low-dose clobetasol, to the affected area of the skin once or twice daily up to about two weeks; wherein said composition provides a low percentage of reduction in DHEAS. In some embodiments, low-dose clobetasol is about 0.025% (w/w) clobetasol. In some embodiments, low-dose clobetasol is about 0.025% (w/w) clobetasol propionate.

In some embodiments, the present invention relates to methods for the prophylaxis, amelioration, or treatment of psoriasis, relief of the inflammatory and pruritic manifestations of steroid responsive dermatoses, erythema, contact sensitivity reactions, and other associated diseases or disorders, by administering to a pediatric subject, comprising administration a low-dose of clobetasol to the affected area of the skin once or twice daily up to about four weeks; wherein said composition provides a low percentage of reduction in DHEAS. In some embodiments, low-dose clobetasol is about 0.025% (w/w) clobetasol. In some embodiments, low-dose clobetasol is about 0.025% (w/w) clobetasol propionate.

Some embodiments are directed to methods of treating psoriasis in a pediatric subject, said method comprising the topical administration of a composition comprising a low-dose of clobetasol to affected areas of the skin once or twice daily for at least one day to about two weeks; wherein said composition is substantially free of adverse effects. In some embodiments, low-dose clobetasol is about 0.025% (w/w) clobetasol. In some embodiments, low-dose clobetasol is about 0.025% (w/w) clobetasol propionate.

In other embodiments, the present invention also provides a process for preparing a topical pharmaceutical composition for use in a pediatric subject, comprising:
(i) preparing an oil phase by melting and stirring thickening agent(s), emulsifier(s) followed by preservative(s) and emollient(s);
(ii) preparing an aqueous phase by heating water,
(iii) preparing an emulsion by adding the oil phase of step (i) to the aqueous phase of step (ii) or vice versa under constant homogenization,
(iv) dissolving a premixed solution of clobetasol in a solvent followed by addition of an antioxidant(s) and homogenizing to obtain a clobetasol solution, and
(v) adding the clobetasol solution obtained in step (iv) to the emulsion prepared in step (iii) followed by homogenization and cooling to obtain a cream composition.

In still further embodiments, the present invention provides a process for preparing a topical pharmaceutical composition for use in a pediatric subject comprising:
(i) preparing an oil phase by melting and stirring stearyl alcohol, cetyl alcohol, white wax, glyceryl stearate and PEG 100 stearate and emollient, followed by methyl paraben and propyl paraben, and the remaining part of the mineral oil,
(ii) preparing an aqueous phase by adding sorbitol solution into heated water,
(iii) preparing an emulsion by adding the oil phase of step (i) to the aqueous phase (ii) or vice versa under homogenization, dissolving a premixed solution of clobetasol propionate in a diethylene glycol monoethyl ether and the followed by addition of BHT and homogenizing to obtain a clobetasol propionate solution, and
(iv) adding the steroid solution obtained in step (iv) to the emulsion prepared in step (iii) followed by homogenization to obtain a cream composition.

In further embodiments, the compositions of the present invention using one or more other corticosteroids can be prepared by using a process similar to that described above. The topical pharmaceutical composition of the present invention is useful in the prophylaxis, amelioration or treatment of skin diseases or disorders such as psoriasis/psoriatic plaques, relief of the inflammatory and pruritic manifestations of steroid responsive dermatoses, erythema, contact sensitivity reactions, atopic dermatitis, seborrheic dermatitis, eczema, plaque psoriasis, erythrodermic psoriasis, psoriasis of the scalp, and other associated diseases or disorders in pediatric subjects. In some embodiments, of the present invention, it was surprisingly found that the topical compositions of the invention containing an oil phase that comprises at least one penetration enhancing agent, and an aqueous phase, provides an enhanced flux of clobetasol through the localized region of the body surface to reach the dermis layer; this advantageously allows for the use of a lower concentration of clobetasol, while providing no significant effect on the endocrine system i.e., HPA axis suppression in pediatric subjects. In some embodiments, of the invention, the pharmaceutical compositions of the present invention may contain 3% of a penetration enhancing agent. In further embodiments of the invention, the pharmaceutical compositions of the present invention may contain 10% of a penetration enhancing agent. In some embodiments, administration of the topical compositions results in HPA axis suppression in less than about 15% of the pediatric subjects treated. In some embodiments, administration of the topical compositions results in HPA axis suppression without any clinical symptoms.

### EXAMPLES

The present invention is illustrated below by reference to the following examples. However, one skilled in the art will appreciate that the specific methods and results discussed are merely illustrative of the present invention, and not to be construed as limiting the application. The following examples may include compilations of data that are representative of data gathered at various times during the course of development and experimentation related to the present invention.

The following general manufacturing processes were followed to prepare Examples 1-7:

Manufacturing Process I:
a) preparing an oil phase by melting and stirring stearyl alcohol, cetyl alcohol; glyceryl stearate and PEG 100 stearate and lanolin followed by methyl paraben and propyl paraben, and mineral oil,
b) preparing an aqueous phase by adding sorbitol solution into heated purified water,
c) preparing an emulsion by adding the oil phase of step (i) to the aqueous phase (ii) or vice versa under homogenization,
d) dissolving a premixed solution of clobetasol propionate in a diethylene glycol monoethyl ether and the followed by addition of BHT and homogenized to obtain a clobetasol propionate solution,
e) adding the clobetasol propionate solution obtained in step (iv) to the emulsion prepared in step (iii) followed by homogenization to obtain a cream composition.

Manufacturing Process II:
a) preparing an oil phase by melting and stirring cetosteryl alcohol; white wax; glyceryl stearate and PEG 100 stearate and isopropyl myristate followed by methyl paraben and propyl paraben, and cyclomethicone,
b) preparing an aqueous phase by heating the purified water,
c) preparing an emulsion by adding the oil phase of step (i) to the aqueous phase (ii) or vice versa under homogenization,
d) dissolving a premixed solution of clobetasol propionate in a diethylene glycol monoethyl ether and the followed by addition of BHT and homogenized to obtain a clobetasol propionate solution,
e) adding the clobetasol propionate solution obtained in step (iv) to the emulsion prepared in step (iii) followed by homogenization to obtain a cream composition.

### Example 1: Clobetasol Propionate (0.05% (w/w)) Cream

A composition of the present example was prepared by following Manufacturing Process-I using the following ingredient amounts.

| Ingredient | Percentage (w/w) |
|---|---|
| Clobetasol propionate | 0.05 |
| Stearyl alcohol | 2 |
| Cetyl alcohol | 2 |
| Glyceryl stearate & PEG 100 | 7.5 |
| Lanolin | 2 |
| Mineral oil | 5 |
| Sorbitol solution, 70% | 2 |
| Diethylene glycol monoethyl ether | 3 |
| Butylated hydroxy toluene | 0.05 |
| Methyl paraben | 0.2 |
| Propyl paraben | 0.4 |
| Purified water | q.s to 100 |

### Example 2: Clobetasol Propionate (0.025% (w/w)) Cream

A composition of the present example was prepared by following Manufacturing Process-I using the following ingredient amounts.

| Ingredient | Percentage (w/w) |
|---|---|
| Clobetasol propionate | 0.025 |
| Stearyl alcohol | 2 |
| Cetyl alcohol | 2 |
| Glyceryl stearate & PEG 100 stearate | 7.5 |
| Lanolin | 2 |
| Mineral oil | 5 |
| Sorbitol solution, 70% | 2 |
| Diethylene glycol monoethyl ether | 10 |
| Butylated hydroxy toluene | 0.05 |
| Methyl paraben | 0.2 |
| Propyl paraben | 0.4 |
| Purified water | q.s to 100 |

### Example 3: Clobetasol Propionate (0.025% (w/w)) Cream

A composition of the present example was prepared by following Manufacturing Process-I using the following ingredient amounts.

| Ingredient | Percentage (w/w) |
|---|---|
| Clobetasol propionate | 0.025 |
| Stearyl alcohol | 2 |
| Cetyl alcohol | 2 |
| Glyceryl stearate & PEG 100 stearate | 7.5 |
| Lanolin | 2 |
| Mineral oil | 5 |
| Sorbitol solution, 70% | 2 |
| Diethylene glycol monoethyl ether | 3 |
| Butylated hydroxy toluene | 0.05 |
| Methyl paraben | 0.2 |
| Propyl paraben | 0.4 |
| Purified water | q.s to 100 |

### Example 4: Clobetasol Propionate (0.025% (w/w)) Cream

A composition of the present example was prepared by following Manufacturing Process-I using the following ingredient amounts.

| Ingredient | Percentage (w/w) |
|---|---|
| Clobetasol propionate | 0.025 |
| Stearyl alcohol | 1.5 |
| Cetyl alcohol | 2.5 |
| Glyceryl stearate & PEG 100 stearate | 7.5 |
| Lanolin | 3 |
| Mineral oil | 4 |
| Sorbitol solution, 70% | 2 |
| Diethylene glycol monoethyl ether | 5 |
| Butylated hydroxy toluene | 0.05 |
| Methyl paraben | 0.2 |
| Propyl paraben | 0.4 |
| Purified water | q.s to 100 |

### Example 5: Clobetasol Propionate (0.025% (w/w)) Cream

A composition of the present example was prepared by following Manufacturing Process-II using the following ingredient amounts.

| Ingredient | Percentage (w/w) |
|---|---|
| Clobetasol propionate | 0.025 |
| Cetosteryl alcohol | 3 |
| Glyceryl stearate & PEG 100 stearate | 6 |
| White wax | 1 |
| Diethylene glycol monoethyl ether | 3 |
| Butylated hydroxytoluene | 0.05 |
| Isopropyl myristate | 10 |
| Cyclomethicone | 5 |
| Methyl paraben | 0.2 |
| Propyl paraben | 0.4 |
| Purified water | q.s to 100 |

### Example 6: Clobetasol Propionate (0.025% (w/w)) Cream

A composition of the present example was prepared by following Manufacturing Process-II using the following ingredient amounts.

| Ingredient | Percentage (w/w) |
|---|---|
| Clobetasol propionate | 0.025 |
| Cetosteryl alcohol | 3 |
| Glyceryl stearate & PEG 100 stearate | 6 |
| White wax | 1 |
| Diethylene glycol monoethyl ether | 10 |
| Butylated hydroxy toluene | 0.05 |
| Isopropyl myristate | 10 |
| Cyclomethicone | 5 |
| Methyl paraben | 0.2 |
| Propyl paraben | 0.4 |
| Purified water | q.s to 100 |

### Example 7: Clobetasol Propionate (0.05% (w/w)) Cream

A composition of the present example was prepared by following Manufacturing Process-II using the following ingredient amounts.

| Ingredient | Percentage (w/w) |
|---|---|
| Clobetasol propionate | 0.05 |
| Cetosteryl alcohol | 3 |
| Glyceryl stearate & PEG 100 stearate | 6 |
| White wax | 1 |
| Diethylene glycol monoethyl ether | 10 |
| Butylated hydroxy toluene | 0.05 |
| Isopropyl myristate | 10 |
| Cyclomethicone | 5 |
| Methyl paraben | 0.2 |
| Propyl paraben | 0.4 |
| Purified water | q.s to 100 |

### Example 8: An Open Label, Multicenter Study to Assess the Potential for Adrenal Suppression and Systemic Drug Absorption Following Multiple Dosing with DFD-06 (Clobetasol Propionate Cream, 0.025%) in Pediatric Subjects with Moderate to Severe Plaque Psoriasis

Subject Population: 21 subjects ages 12-16 completed the study. Per study entry criteria all subjects had psoriasis IGA>=3 and >10% BSA.

Treatment: DFD-06 (Clobetasol cream, 0.025%) BID for 15 days (at least 5 g/day). Notably there was no maximum dose limitation in the study.

Adverse Events included: vein collapse—mild, not related; 3 cases of itching—mild, probably related; Burning/stinging—mild, probably related; 3 cases of HPA suppression—mild, related (14.3%, 3/21 subjects).

Compliance with treatment was based on diary reports. 637 of 648 dose days were reported as completed (98.3%). Additionally, study drug exposure was captured by weights or returned study drug.

PK data: 58 of 63 samples BLQ (10 pg/mL). Three subjects evidenced adrenal suppression (reserve) as measured by the ACTH stimulation test; all had detectable plasma clobetasol at the two week on treatment visit.

ACTH Stimulation Test: Cortisol levels <18 mcg/dL post ACTH stimulation were considered as demonstrating suppression. Three subjects evidenced adrenal suppression as determined by the ACTH stimulation test on study day 15 following two weeks of twice daily treatment. None of the subjects had clinical signs or symptoms of HPA axis suppression.

Subject 101-001, age 14 with 24% BSA involved with psoriasis had exposure to ˜95 g study drug/2 weeks. The subject had a within normal range morning cortisol on day 15 (8.1 mcg/dL). Post stimulation cortisol was 16 mcg/dL. Plasma clobetasol was not detectable prior to the day 15 dose and was just above the LLQ (11 pg/mL) after day 15 dosing. At one-month follow-up after cessation of study drug the ACTH stimulation results were normal.

Subject 105-003, age 16.4 with 16% BSA involved with psoriasis had exposure to 289 g study drug/2 weeks. The subject had an abnormal morning cortisol on day 15 (0.9 mcg/dL). Post stimulation cortisol was also low (7.5 mcg/dL). The subject also had detectable plasma clobetasol prior to day 15 dose, 156 pg/mL with a peak plasma concentration of 343 pg/mL one hour post the day 15 dose. At one-month follow-up after cessation of study drug the stimulation results were normal.

Subject 201-010, age 12.5 years completed the day 15 ACTH stimulation test. The subject did not return study drug for evaluation of amount of study drug applied. Day 15 pre-stimulation cortisol level was within normal limits, 6.2 mcg/dL and the post stimulation was low at 12.4 mcg/dL. Plasma clobetasol was detectable pre-dose Day 15 (26.1 pg/mL) with a peak post dose level that day of 29.8 pg/mL. No symptoms suggestive of adrenal suppression were reported and the subject and parent declined further evaluations or follow-up visits.

Discussion: The rate of HPA suppression in subjects age 12-16 was 14.3% (3/21 subjects). This rate was consistent with the adult population (12.5%, 3/24) treated in study DFD-006-007. Study DFD-006-007 treated adult subjects with DFD-06 (Clobetasol cream, 0.025%) BID for 15 days (˜5 g-7/day) who had psoriasis IGA>=3 and 20-50% BSA. Temovate (Clobetasol cream, 0.05%) in the 007 Study had a rate of HPA suppression of 36.4% (8/22 subjects).

Notably all three of the subjects who evidenced abnormal ACTH stimulation had detectable plasma clobetasol after 2 weeks of twice daily dosing. None of these subjects had other clinical signs or symptoms of HPA axis suppression. For one of these three subjects the plasma clobetasol was just above the limits of detection of the assay. This subject was exposed to ˜47 gms of product per week. Per the USPI for the adult population, no more than 50 gms of product should be applied per week. The second subject with abnormal ACTH stimulation had detectable plasma clobetasol both with the final dose and prior to that dose, suggesting ongoing plasma clobetasol exposure throughout the dosing period. This subject applied approximately 150 gms of product per week. Nonetheless both subjects demonstrated normal ACTH stimulation after cessation of treatment. The third subject with abnormal ACTH stimulation did not return the provided product tubes for assessment of exposure. However, the subject had detectable plasma clobetasol on Day 15 suggesting that the subject had continued with treatment during study days. This subject/parent declined return post treatment to repeat the ACTH test to assess normalization.

Conclusion: Per the study protocol, continuation with the younger age cohort (ages 6-<12 years) can proceed provided the HPA axis suppression rate is <40%. The rate of 14.3% falls well within that threshold. There were no other safety signals that would prevent initiating the younger cohort. However, given the restrictions on maximum product to be applied per USPI and the results the above, no more than 50 gms/week at the most should be applied to any subjects in this age group.

While several particular forms of the application have been illustrated and described, it will be apparent that various modifications and combinations of the application detailed in the text can be made without departing from the spirit and scope of the application.

All publications, patents, and patent applications mentioned in this specification are herein incorporated by reference to the same extent as if each individual publication, patent, or patent application was specifically and individually indicated to be incorporated by reference.

What is claimed is:

1. A method comprising topically administering twice daily for up to about two weeks to a pediatric subject with moderate to severe plaque psoriasis a composition comprising:
about 0.005% to about 0.045% (w/w) of clobetasol;
at least 60% (w/w) of an aqueous phase comprising water;
an oil phase;
about 5% of an emollient;
about 0.05% of an antioxidant;
about 10% of a solvent;
about 6% of an emulsifier; and
a preservative;
wherein the oil phase comprises at least one penetration enhancing agent in an amount from about 0.01% to about 15% of the total weight of the composition and a non-polymeric thickening agent;
wherein the pediatric subject has a risk of HPA axis suppression that is similar to the risk of HPA axis suppression in an adult subject.

2. The method of claim 1, wherein the pediatric subject has moderate to severe plaque psoriasis involving at least about 10% body surface area.

3. The method of claim 1, wherein the pediatric subject has an investigator global assessment score of greater than, or equal to 3.

4. The method of claim 1, wherein the moderate to severe plaque psoriasis is treated in the subject and results in an investigator global assessment score of 0 to 1.

5. The method of claim 4, wherein the investigator global assessment score is measured at day 15.

6. The method of claim 1, wherein the pediatric subject is a male or female aged about 6 to about 16 years and 11 months of age.

7. The method of claim 1, wherein the pediatric subject is a male or female aged about 12 to about 16 years and 11 months of age.

8. The method of claim 1, wherein the pediatric subject is a male or female under the age of 18 years.

9. The method of claim 1, wherein the pediatric subject is a male or female under the age of 12 years.

10. The method of claim 1, wherein topical administration of the composition to the pediatric subject results in an at least 80% chance that the subject will not develop hypothalamic-pituitary-adrenal (HPA) axis suppression.

11. The method of claim 1, wherein topical administration to the pediatric subject results in the subject being substantially free of adverse effects.

12. The method of claim 11, wherein the adverse effect is selected from skin irritation, vein collapse, itching, burning, stinging and a combination thereof.

13. The method of claim 11, wherein the adverse effect is selected from striae and skin atrophy or a combination thereof.

14. The method of claim 11, wherein the adverse effect is selected from Cushing's syndrome, hyperglycemia, and glycosuria, linear growth retardation, delayed weight gain, and intracranial hypertension and a combination thereof.

15. The method of claim 1, wherein topical administration to the pediatric subject results in no hypothalamic-pituitary-adrenal (HPA) axis suppression.

16. The method of claim 1, wherein topical administration to the pediatric subject results in substantially no hypothalamic-pituitary-adrenal (HPA) axis suppression.

17. The method of claim 1, wherein topical administration to the pediatric subject results in plasma concentrations of clobetasol that are insufficient to reduce serum cortisol levels less than or equal to 18 μg/dL.

18. The method of claim 1, wherein the clobetasol is clobetasol propionate.

19. The method of claim 1, wherein the topical administration of the composition provides a mean clobetasol plasma level less than about 10 pg/mL.

20. The method of claim 1, wherein the emollient comprises cyclomethicone

21. The method of claim 1, wherein the antioxidant comprises butylated hydroxytoluene.

22. The method of claim 1, wherein the solvent comprises isopropyl myristate.

23. The method of claim 1, wherein the emulsifier comprises glyceryl stearate and PEG 100.

24. The method of claim 1, wherein the preservative comprises one or both of methylparaben and propylparaben.

25. The method of claim 24, wherein the preservative is methylparaben and the methylparaben is about 0.2% of the total weight of the composition.

26. The method of claim 24, wherein the preservative is propylparaben and the propylparaben is about 0.4% of the total weight of the composition.

27. The method of claim 1, wherein the penetration enhancing agent is diethylene glycol monoethyl ether.

28. The method of claim 1, wherein the non-polymeric thickening agent comprises one or both of cetosteryl alcohol and white wax.

29. The method of claim 1, wherein the composition is substantially free of propylene glycol.

30. The method of claim 1, wherein the composition is substantially free of polymers.

31. The method of claim 1, wherein the oil phase comprises at least one penetration enhancing agent in an amount up to about 5.0% of the total weight of the composition, wherein the at least one penetration enhancing agent is diethylene glycol monoethyl ether.

32. The method of claim 1, wherein the oil phase comprises at least one penetration enhancing agent in an amount of about 3.0% of the total weight of the composition, wherein the at least one penetration enhancing agent is diethylene glycol monoethyl ether.
